(19) United States
(12) Patent Application Publication
(10) Pub. No.: US 2026/0209664 A1
(43) Pub. Date: Jul. 23, 2026

(54) A MODULAR SCALE-DOWN REACTOR

(71) Applicant: Danmarks Tekniske Universitet, Kongens Lyngby (DK)

(72) Inventors: Suresh Sudarsan, Kongens Lyngby (DK); Jan Frank Pedersen, Kongens Lyngby (DK)

(21) Appl. No.: 19/137,762

(22) PCT Filed: Dec. 15, 2023
(86) PCT No.: PCT/EP2023/085993

§ 371 Date: Jun. 11, 2025

(30) Foreign Application Priority Data
Dec. 16, 2022 (EP) 22214162.4

(51) Int. Cl. C12M 3/00; C12M 1/02; C12M 1/06; C12M 1/36

(52) U.S. Cl. CPC C12M 23/44; C12M 27/02; C12M 41/18; C12M 41/48

(57) ABSTRACT

The invention relates to a modular scale-down reactor comprising two or more reversibly attached reactor units, one or more inlet apertures, one or more outlet apertures, and at least one disk to be inserted between two adjacent reactor units or within an inner reaction unit. The invention further relates to the use of said scale-down reactor for simulating, modelling, monitoring or modifying the conditions in a large-scale reactor.

### FIELD OF INVENTION

The invention relates to a modular scale-down reactor.

### BACKGROUND

Reactors for bio-based production processes are commonly used in the biotech and biochemical industry for production of a range of compounds, spanning from recombinant antibodies and biopharmaceuticals to industrial enzymes and biochemicals. Commonly, bio-based production processes rely on cultivation or fermentation of microorganisms or mammalian cells, or on enzymatic processes, in large-scale or pilot scale reactors of 300 L to >100,000 L. Stirred tank reactors (STRs) and bubble column reactors (BCRs) are two of the most commonly used reactor types.

The successful transfer of a bio-based production process from laboratory scale (small-scale) (0.25-30 L) to pilot plant and industrial scale (large-scale) (100 L to >100,000 L) depends on solving the scale-dependent challenges that are typically associated with scaling up. In particular, due to the large volumes employed, various gradients, i.e., heterogeneity, occur due to mixing challenges resulting from fluctuating mixing times in different parts of the large-scale or pilot scale reactor. This affects cell growth and survival, and thereby the production of metabolites of interest synthesised by the cells.

At large-scale there may be gradients present with fluctuating oxygen availability, substrate/nutrient availability, metabolite concentration and pH, which can result in inhibition of cell growth and production. A limited understanding of these challenges and how they affect cell/microbial physiology during scale-up means that production organisms may perform poorly when tested in large-scale conditions, even though said cell/microorganism performs well at small-scale.

Thus, various scale-up strategies can be and have been applied in order to manage successful transfer of the production process from small to large-scale, and to better predict the performance of a bio-based production process at large-scale (Junker, 2004). In particular, various so-called scale-down approaches and reactors have been developed. Using such approaches and reactors, one can generate sufficient data indicating how to build and/or how to change/optimize the large-scale process (Haringa, 2019; Neubauer and Junne, 2010; Oosterhuis 1984). Hence, experiments in scale-down reactors are often helpful and may even be necessary to ensure rapid process development and successful transfer of a bioproduction process to large-scale.

Thus, scale-down reactors can be used for developing a large-scale bioproduction process with an optimized design and performance. A scale-down reactor can for instance be used to:
i. simulate, model and/or mimic large-scale reactors in terms of the large-scale dynamic environment with gradients, heterogeneity and zones with fluctuating mixing times. The information gained from the scale-down reactor can be used to predict, identify and/or address challenges at large-scale; and/or
ii. evolve strains that are more robust and better tolerate the above-described large-scale environment.

Scale-down reactors have been previously described. Schilling et al. and Gaugler et al. disclose systems in which a single reactor chamber can be subdivided in different zones by using different inlets. However, the size of the reactor of said disclosures is not adjustable in itself. WO12097079 discloses a system in which the working volume is adjusted by changing the overall volume of the reactor before construction or before starting the fermentation, e.g., by taking advantage of an inner chamber or bag, the volume of which can be adjusted before the reaction, and which can be inserted in a non-modular reactor housing of fixed dimensions. Thus, in said disclosure, the size of the reactor compartment is adjustable, but not in a modular way. Rather, the reactor housing has a constant size, and can accommodate bioprocessing bags of different sizes, but only one bag at the time.

There is thus a need for versatile and adjustable scale-down reactors, the configuration of which can be easily modified to mimic a plethora of conditions.

### SUMMARY OF THE INVENTION

The invention disclosed herein relates to a scale-down reactor composed of modules (i.e., units). In particular, the present invention relates to a scale-down reactor comprising:
a. a reaction compartment comprising at least two reactor units, wherein said at least two reactor units are reversibly attached to one another via connection means,
   wherein the at least two reactor units comprise a top unit and a bottom unit, and optionally one or more further inner reactor units;
   wherein said reactor units when attached to one another form a fluid-tight reaction compartment for receiving or configured to receive a reaction liquid therein;
b. one or more inlet apertures,
c. one or more outlet apertures,
d. optionally, a mixing shaft placed or configured to be placed vertically in the reaction compartment, said mixing shaft comprising one or more impellers, and
e. at least one disk inserted or configured to be inserted horizontally between two adjacent reactor units or horizontally within an inner reaction unit, said disk comprising at least one aperture allowing the fluid contained in the adjacent reactor units to pass through it, optionally said disk comprising at least one shaft aperture allowing the mixing shaft to pass through it.

As indicated above, the modules/chambers are designed to be attached or detached to each other in a fluid-tight manner, allowing to easily adjust the effective volume of the scale-down reactor. Inlets in the form of disks with various designs can be inserted between the modules to create different gradients. In particular, this feature allows to generate different sub-volumes on the reactor, each of the sub-volumes having a volume an integer multiple of the volume of a single module chamber. Depending on the configuration of the disk separating the compartments of adjacent modules, it is possible to control the level of interaction between the liquids of said adjacent modules and to generate various gradients. This allows the user to, for example, generate sub-volumes with various mixing times and makes it easy to adjust the size and overall properties of a given reactor depending on the experiment, thereby allowing many different conditions and scale-up models to be tested. It is thus possible to test, in a relatively small volume, the challenges that can be expected in large-scale, e.g. gradients arising due to heterogeneous mixing in large volumes. In other words, the scale-down reactor described herein represents a valuable tool to simulate the conditions of poor mixing and inhomogeneous substrate distribution in bioreactors of industrial scale.

Thus, one aspect of the present invention provides a method of simulating, modelling, monitoring or modifying the conditions of a reactor comprising a liquid composition in a bioreactor or live fermentation process, comprising the steps of:
a. providing the scale-down reactor as described herein;
b. defining the total reactor volume by reversibly adding a number of inner reactor units (3);
c. defining the number, position and design of the at least one disk (7); and
d. optionally, defining the design of the mixing shaft (5) and one or more impeller (6);
wherein the conditions to be simulated, modelled, monitored or modified are selected from the group consisting of: reaction volume, temperature, pH, gas concentration, metabolite concentration, cellular mass, cellular density, enzymatic kinetics, absorbance, and mixing time, and wherein the reactor is at least 300 L.

The reactor comprises also a top unit (1) and a bottom unit (2) as described herein.

The scale-down reactor thus can be used to simulate, model, monitor, modify or mimic the conditions expected at large-scale, i.e., in a reactor of at least 300 L.

One aspect of the present invention provides the use of the scale-down reactor described herein for simulating, modelling, monitoring or modifying the conditions of a reactor, wherein the conditions to be simulated, modelled, monitored or modified are selected from the group consisting of: reaction volume, temperature of the fluid in each sub-volume reactor unit, pH in each sub-volume reactor unit, gas concentration, metabolite concentration, cellular mass, cellular density, enzymatic kinetics, absorbance, and mixing time, wherein the reactor is at least 300 L.

The scale-down reactor presented herein is especially suitable for bioproduction processes, i.e., for the culture of bacterial, yeast or mammalian cells for the production of bio-based compounds such as, but not limited to, biopharmaceuticals, biochemicals and enzymes. The scale-down reactor can thus be used for simulating, modelling, monitoring or modifying the conditions of chemical reactions, enzymatic reactions and/or biological reactions. The scale-down reactor can in particular be used to evolve a cell or microorganism that is more robust to the environment and gradients of a large-scale reactor. Thus, in some aspects, the present invention provides a method of adapting a microorganism to the conditions of a reactor, said method comprising:
a. providing the scale-down reactor described herein;
b. defining the total reactor volume by reversibly adding a number of inner reactor units;
c. defining the number, position and design of the at least one disk;
d. optionally, defining the design of the mixing shaft and one or more impellers; and
e. growing the microorganism in the scale-down reactor for an extended amount of time in order to allow the microorganism to adapt to the conditions of the reactor;
wherein the reactor is at least 300 L, thereby obtaining an adapted microorganism.

### DESCRIPTION OF THE DRAWINGS

FIG. 1 shows, from left to right:
an inner reactor unit (3);
a mixing shaft (5) with impellers (6);
an example set-up of the scale-down reactor in exploded view with a top unit (1) with an observation aperture (9) and several inlet/outlet apertures (4), four inner reaction units (3) with inlet/outlet apertures (4), and a bottom unit (2) with two inlet/outlet apertures (4); and
the assembled example set-up described above.

FIG. 2 shows, from left to right:
an inner reactor unit (3) with a disk (7) with a shaft aperture (8) and several additional apertures of different size (16); and
an example set-up of the scale-down reactor in exploded view with a with a top unit (1) with an observation aperture (9) and several inlet/outlet apertures (4), four inner reaction units (3) with inlet/outlet apertures (4), wherein each inner reaction unit comprises a disk (7) with a shaft aperture (8) and several additional apertures; and a bottom unit (2) with two inlet/outlet apertures (4).

FIG. 3 shows a top unit (1) with an observation aperture (9).

FIG. 4 shows a bottom unit (2) from two different angles, said bottom unit comprising several inlet apertures for injection of gases (17) and two inlet/outlet apertures (4).

FIG. 5 shows an observation aperture (9).

FIG. 6 shows (top figure) an example of an assembled temperature control jacket (10) comprising a top jacket unit (11), a bottom jacket unit (12) and several middle jacket units (13), as well as a jacket inlet aperture (14) and a jacket outlet aperture (15). The lower figure shows the bottom jacket unit alone.

FIG. 7 shows a top jacket unit (11).

FIG. 8 shows a bottom jacket unit (12).

FIG. 9 shows a middle jacket unit (13).

FIG. 10 shows a middle jacket unit (13) with a jacket inlet/outlet aperture (14/15).

FIG. 11 shows a disk (7) with a shaft aperture (8) and several additional apertures of different size (16).

FIG. 12 shows a disk (7) with a shaft aperture (8) and several additional apertures of different size (16).

FIG. 13 shows a disk (7) with a shaft aperture (8) and three additional apertures (16).

FIG. 14. Growth profile of E. coli in the reference configuration of scale-down reactor. The top figure (FIG. 14) shows the biomass concentration increase in a batch cultivation measured from the samples withdrawn manually from the reactor's bottom sampling port. The bottom figure (FIG. 14, continued) shows the real-time online data (dissolved oxygen pO₂, stirrer speed, temperature, pH and CO₂ and Oxygen measured in the reactor off-gas) monitored using the control system during cultivation.

FIG. 15. Growth profile of E. coli in the scale-down reactor. The top figure (FIG. 15) shows the biomass concentration increase in a batch cultivation measured from the samples withdrawn manually from the reactor's bottom sampling port. The bottom figure (FIG. 15, continued) shows the real-time online data (dissolved oxygen pO₂, stirrer speed, temperature, pH and CO₂ and Oxygen measured in the reactor off-gas) monitored using the control system during cultivation.

FIG. 16. Liquid mixing time obtained in the scale-down reactor with different types of modular chambers and with 3 Rushton impellers (FIG. 16A) and 3/6 impellers (FIG. 16B), in comparison with the industrial large-scale stirred tank reactors (in m³ volume) mixing time reported from literature (FIG. 16C).

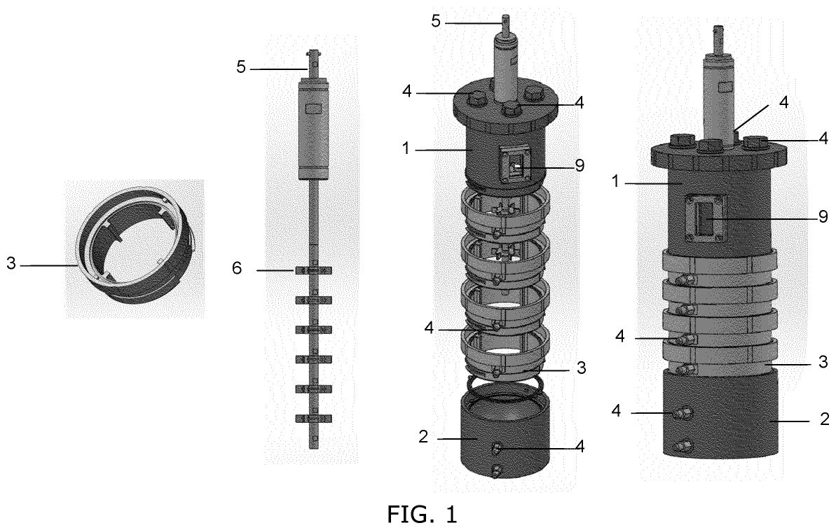

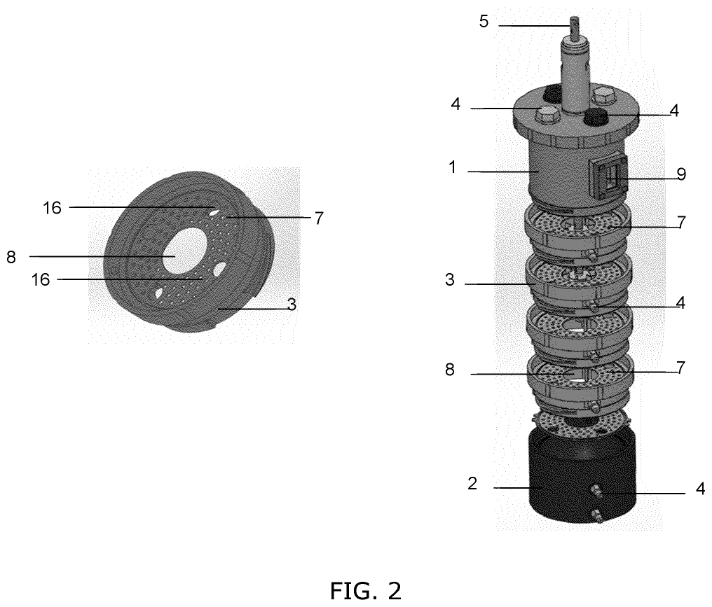

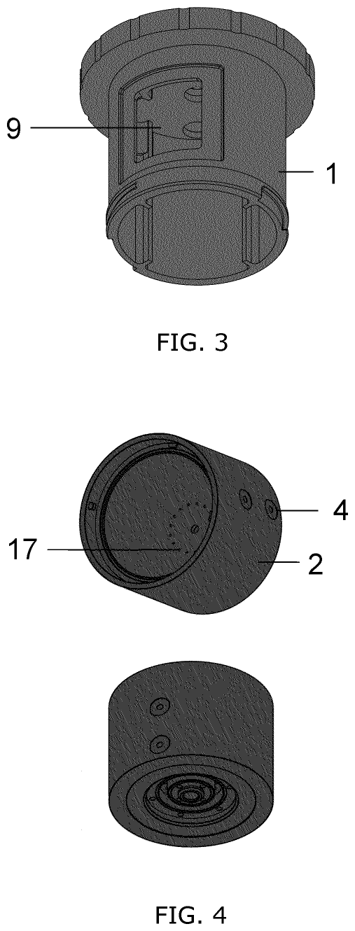

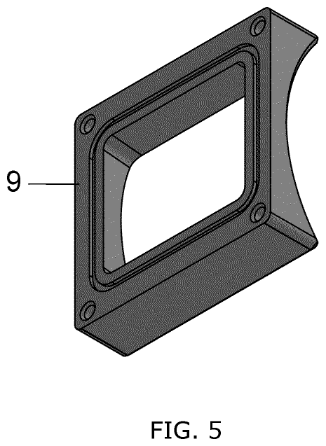

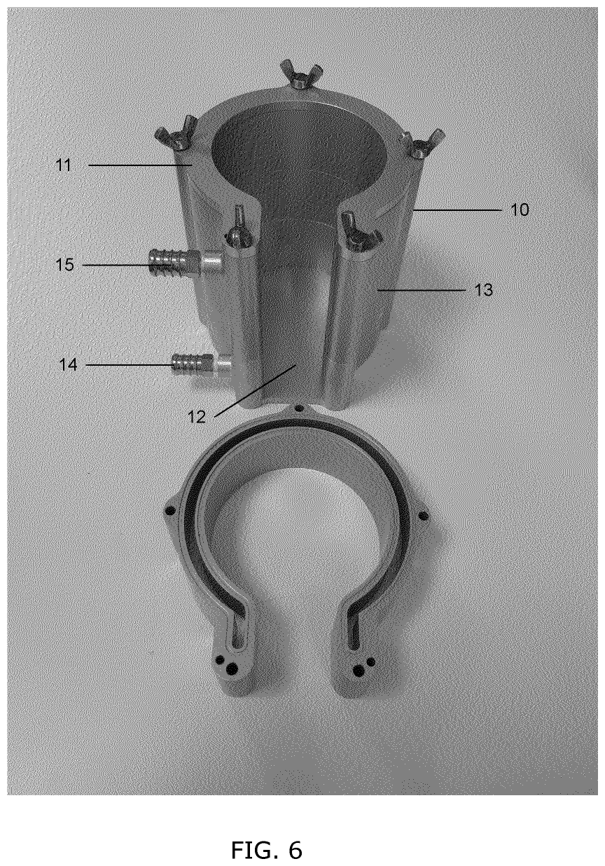

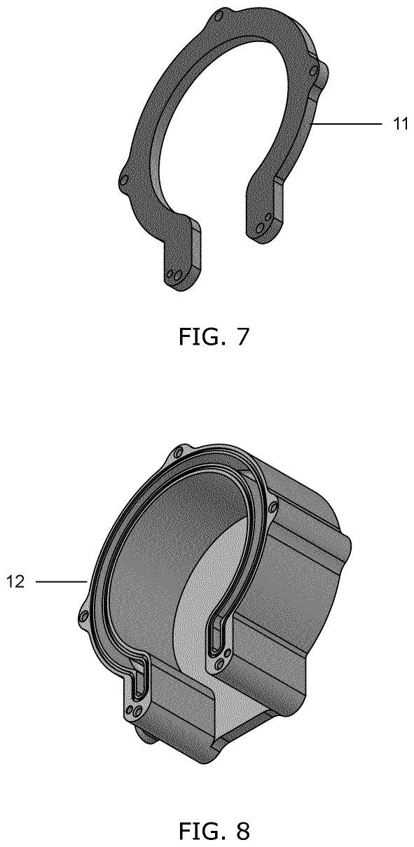

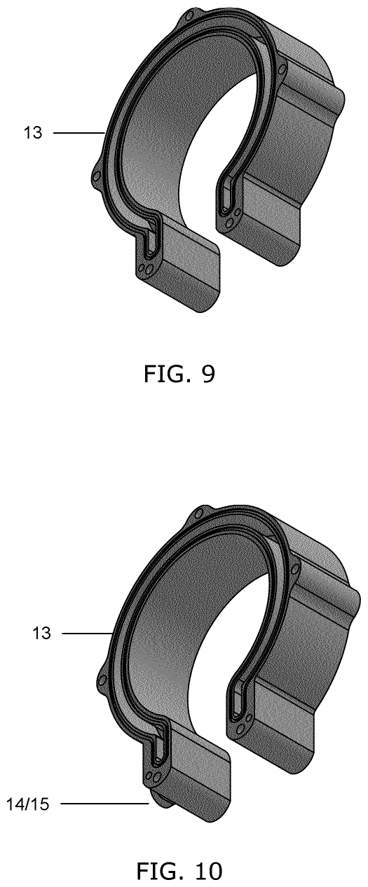

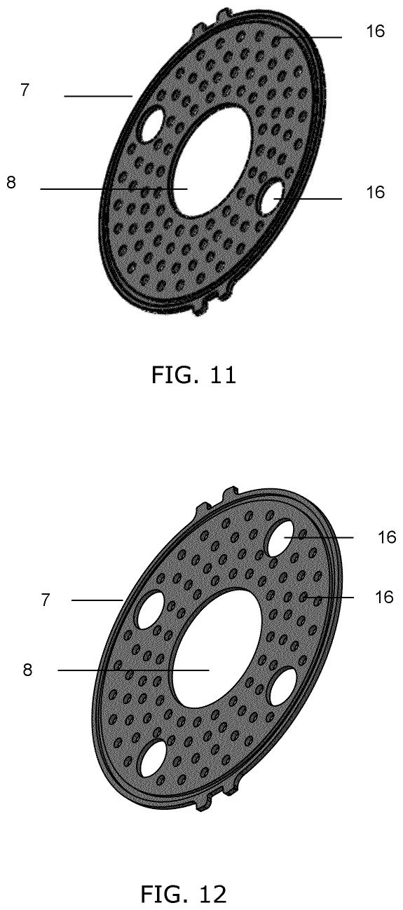

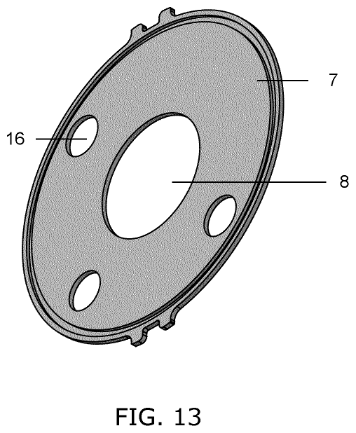

### DETAILED DESCRIPTION

### Definitions

‘Adjacent reactor units’ refers herein to any two reactor units which are located next to one another. In an assembled configuration of the scale-down reactor, these units are connected via connection means allowing a fluid-tight separation between the inner volume contained in said units and the outer environment of the scale-down reactor, while allowing fluid to pass from one unit to the next. Adjacent reactor units can refer to:
the top unit and the inner reactor unit adjacent to it;
two inner reactor units; or
the bottom unit and the inner reactor unit adjacent to it.

‘Aspect ratio’ as used herein is the inner diameter of a reactor divided by the inner height of the same reactor.

‘Baffles’ as used herein refers to flow-directing or obstructing vanes or panels used to direct a flow of liquid or gas. The baffles of a reactor are commonly placed on the inner wall of the reactor.

‘Bubble column reactor’ as used herein refers to a type of reactor in which gas is inserted at the bottom and bubbles up through the reaction liquid. Mixing is provided by the upward velocity of the bubbles.

‘Conditions’ as used herein refers to, but is not limited to, conditions of a reactor or scale-down reactor such as reaction volume (i.e. volume of the reaction liquid), temperature, pH, gas concentration, metabolite concentration, cellular mass, cellular density, enzymatic kinetics, absorbance, and mixing time.

‘Effective volume’ or ‘total volume’ as used interchangeably herein refers to the total inner volume of a reactor.

‘Fluid’ refers to a liquid or a gas. As will be evident to the skilled person, the fluid-tight reaction compartment of the present scale-down reactor is for or is configured for receiving a reaction liquid and is thus also suited for receiving a reaction gas, in other words the fluid-tight reaction compartment is for or is configured for receiving a reaction gas.

‘Impeller tip speed’ as used herein refers to the speed with which a selected point on the peripheral of the impeller travels in a set time. It can be calculated using the formula:

$$Impeller tip speed = π * D * n$$

wherein D is the impeller diameter and n is the impeller rotation speed.

‘Mixing shaft unit’ as used herein refers to a modular part of the mixing shaft.

‘Mixing time’ as used herein refers to the time required for achieving a pre-determined degree of homogeneity in a reactor or, where indicated, in a sub-volume of a reactor.

‘Operating conditions’ as used herein refers to, but is not limited to, Power input value (P/V), aspect ratio, impeller tip speed, mixing time, volumetric mass transfer coefficient and oxygen transfer rate.

‘Oxygen transfer rate’ or OTR as used herein refers to the transfer rate of oxygen. Together with the volumetric mass transfer coefficient (k_{L}a), it indicates how efficient oxygen is transferred from the gas bubbles into the reactor liquid. It can be calculated from the formula:

$${k}_{L} a = OTR / ( {C}_{L}^{⋆} - {C}_{L} )$$

wherein k_{L}a is the volumetric mass transfer coefficient, C*_{L} is the dissolved oxygen concentration at air saturation and C_{L} is the dissolved oxygen concentration in the liquid phase.

‘Power input value’ as used herein refers to the Power input divided by the volume and can be calculated according to the formula:

$$P = Po * ρ * N * {D}^{3}$$

wherein V is the volume of the reaction liquid, Po is the Power number for the impeller, ρ is the fluid density of the reaction liquid, N is the rotational speed of the impeller and D is the impeller diameter.

‘Reactor unit’ as used herein refers to a modular part of the scale-down reactor.

‘Sparger’ as used herein refers to any type of device for introducing air in the reactor.

‘Specific exchange area’ as used herein refers to the area of all apertures on a disk. The specific exchange area can be chosen to achieve a specific mixing time of the scale-down reactor.

‘Specific flow profile’ as used herein refers to the flow of reaction liquid through the apertures on the disk(s). The flow profile is determined by the specific exchange area, by the type of mixing used, and, if using a mixer/impeller, by the impeller type and the impeller speed.

‘Stirred tank reactor’ as used herein refers to a reactor equipped with an impeller or other mixing device to provide efficient mixing.

‘Sub-volume’ as used herein refers to the space between two adjacent disks or between one disk and the top or the bottom of the reactor.

‘Volumetric mass transfer coefficient’ or k_{L}a is a measure of the rate of oxygen used for the bioreactor process. Together with the oxygen transfer rate (OTR), it indicates how efficient oxygen is transferred from the gas bubbles into the reactor liquid. It can be calculated from the formula:

$${k}_{L} a = OTR / ( {C}_{L}^{⋆} - {C}_{L} )$$

wherein OTR is the oxygen transfer rate, C*_{L} is the dissolved oxygen concentration at air saturation and C_{L} is the dissolved oxygen concentration in the liquid phase.

‘Working volume’ as used herein refers to the volume reaction liquid comprised in a reactor.

### Modular Scale-Down Reactor

The present invention relates to a scale-down reactor comprising:
a. a reaction compartment comprising at least two reactor units, wherein said at least two reactor units are reversibly attached to one another via connection means,
   wherein the at least two reactor units comprise a top unit (1) and a bottom unit (2), and optionally one or more further inner reactor units (3);
   wherein said reactor units when attached to one another form a fluid-tight reaction compartment for receiving or configured to receive a reaction liquid therein;
b. one or more inlet apertures (4),
c. one or more outlet apertures (4),
d. optionally, a mixing shaft (5) placed or configured to be placed vertically in the reaction compartment, said mixing shaft comprising one or more impellers (6), and
e. at least one disk (7) inserted or configured to be inserted horizontally between two adjacent reactor units or horizontally within an inner reaction unit, said disk comprising at least one aperture (16) allowing the fluid contained in the adjacent reactor units to pass through it, optionally said disk comprising at least one shaft aperture (8) allowing the mixing shaft to pass through it.

The scale-down reactor is particularly useful for simulating, monitoring, modelling and/or mimicking large-scale and/or pilot scale reactors, such as for simulating, monitoring, modelling and/or mimicking gradients that are likely to occur at larger scale. Such gradients may for example arise due to mixing challenges present at larger scale. In particular, the scale-down reactor allows for identification of such mixing challenges prior to scaling up a reactor production process. Very preferably, the scale-down reactor is constructed such that the mixing time is similar or identical to the mixing time of a large-scale or pilot scale reactor. The reaction compartment is fluid-tight and can received, or is configured to receive, a reaction liquid, a reaction gas, or a combination of reaction liquid and reaction gas. Thus the scale-down reactor is useful for simulating, monitoring, modelling and/or mimicking reactions including reactions occurring in a liquid, reactions occurring in a gas, and reactions occurring in a liquid and gas mixture. Thus, throughout the present disclosure, it will be clear that a compartment for receiving, be configured to receive or comprising a reaction liquid may also be for receiving, be configured to receive or comprising a reaction gas, such as a gaseous substrate.

The scale-down reactor is also particularly useful for developing, i.e. evolving, microbial strains that are more tolerant, i.e. more robust, to the dynamic and heterogeneous environment in a large-scale or pilot scale reactor.

The scale-down reactor of the present disclosure may be any type of reactor. Preferably, it is a bioreactor, such as a reactor used for bio-based production processes. Said processes commonly rely on cultivation or fermentation of microorganisms or mammalian cells, or on enzymatic processes. In some embodiments, the scale-down reactor is a stirred tank reactor. In other embodiments, the scale-down reactor is a bubble column reactor.

The scale-down reactor may be manufactured in any material that is suitable for a bioproduction process. Preferably, the scale-down reactor is manufactured from stainless steel or a polymer, even more preferably stainless steel of type 306 or 306 L or polycarbonate.

In one embodiment, the top unit and/or the bottom unit are manufactured from stainless steel, such as stainless steel of type 306 or 306 L, and the one or more further inner reactor units are manufactured from a polymer, such as polycarbonate.

In one embodiment, the polycarbonate has good dimensional stability, low moisture content absorption, high hardness, higher heat deflection temperature, good impact strength and/or high stiffness. In one embodiment, the polycarbonate is Sustanat® PC. In one embodiment, the polycarbonate possesses one or more of the properties listed in the table below.

Table. Sustanat® PC.

| | Test method | Unit | Guideline Value |
|---|---|---|---|
| General properties | | | |
| Density | DIN EN ISO 1183-1 | g / cm³ | 1.20 |
| Water absorption | DIN EN ISO 62 | % | 0.2 |
| Flammability (Thickness 3 mm / 6 mm) | UL 94 | | HB / HB |
| Mechanical properties | | | |
| Yield stress | DIN EN ISO 527 | MPa | 65 |
| Elongation at break | DIN EN ISO 527 | % | 80 |
| Tensile modulus of elasticity | DIN EN ISO 527 | MPa | 2300 |
| Notched impact strength | DIN EN ISO 179 | kJ / m² | 20 |
| Shore hardness | DIN EN ISO 868 | scale D | 82 |
| Thermal properties | | | |
| Thermal conductivity | DIN 52612-1 | W / (m * K) | 0.21 |
| Thermal capacity | DIN 52612 | KJ / (kg * K) | 1.20 |
| Coefficient of linear thermal expansion | DIN 53752 | 10⁻⁶ / K | 70 |
| Service temperature, long term | Average | ° C. | -40 . . . 115 |
| Service temperature, short term (max.) | Average | ° C. | 140 |
| Heat deflection temperature | DIN EN ISO 75, Verf. A, HDT | ° C. | 135 |
| Electrical properties | | | |
| Dielectric constant | IEC 60250 | | 3 |
| Dielectric dissipation factor (50 Hz) | IEC 60250 | | 0.001 |
| Volume resistivity | DIN EN 62631-3-1 | Ω* cm | 10¹⁵ |
| Surface resistivity | DIN EN 62631-3-2 | Ω | 10¹⁵ |
| Comparative tracking index | IEC 60112 | | 275 |
| Dielectric strength | IEC 60243 | kV/mm | 30 |

The scale-down reactor comprises at least a top reaction unit (top unit) and a bottom reaction unit (bottom unit), and at least one disk, such as at least two disks. In some embodiments, the space between two adjacent disks or between one disk and the top or the bottom of the reactor defines a sub-volume from the total volume of the reactor.

The modules, i.e. the reactor units, are designed to be attached or detached to each other in a fluid-tight manner, allowing easily adjusting the effective volume of the scale-down reactor. Fluid can pass from one reactor unit to an adjacent reactor unit, e.g. through the apertures of the disk separating the two adjacent units, but the units are connected to one another in a way that they together define a single, inner volume which is separated from the outer environment of the scale-down reactor in a fluid-tight manner. The scale-down reactor may comprise any feasible number of reactor units.

In some embodiments, the scale-down reactor comprises between 2 and 10 reactor units, such as between 2 and 8 reactor units, such as between 3 and 8 reactor units, such as between 4 and 8 rector units, such as between 5 and 8 reactor units, such as between 6 and 8 reactor units, such as between 3 and 7 reactor units, such as between 4 and 7 reactor units, such as between 5 and 7 reactor units, such as between 2 and 6 reactor units, such as between 3 and 6 reactor units, such as between 4 and 6 reactor units such as between 5 and 6 reactor units, such as between 2 and 5 reactor units, such as between 3 and 5 reactor units, such as between 4 and 5 reactor units, such as between 2 and 4 reactor units, such as between 3 and 4 reactor units, such as between 2 and 3 reactor units.

In some embodiments, the scale-down reactor comprises at least 3 reactor units, such as at least 4 reactor units, such as at least 5 reactor units, such as at least 6 reactor units, such as at least 7 reactor units, such as at least 8 reactor units, such as at least 9 reactor units, such as at least 10 reactor units.

As stated above, the volume of the scale-down reactor is easily adjusted by modifying the number of reactor units. In particular, the total volume of the reactor may be reversibly modified by varying the number of inner reactor units.

In some embodiments, the total volume of the scale-down reactor is between 50 mL and 35 L, such as between 1 L and 35 L, such as between 10 L and 30 L, such as between 10 L and 20 L, such as between 1 L and 10 L, such as between 1 L and 5 L, such as between 100 mL and 10 L, such as between 500 mL and 15 L, such as between 60 mL and 700 mL, such as between 100 mL and 600 mL such as between 50 mL and 500 mL such as between 50 mL and 1 L.

In a particular embodiment, the volume of the scale-down reactor can be adjusted to between 60 mL and 600 mL by modifying the number of reactor units.

In another embodiment, the volume of the scale-down reactor can be adjusted to between 1 L and 10 L by modifying the number of reaction units.

In some embodiments, the total volume of the reactor is at least 50 mL, such as at least 60 mL, such as at least 70 mL, such as at least 80 mL, such as at least 90 mL, such as at least 100 mL, such as at least 200 mL, such as at least 300 mL, such as at least 400 mL, such as at least 500 mL, such as at least 600 mL, such as at least 700 mL, such as at least 800 mL, such as at least 900 mL, such as at least 1 L, such as at least 5 L, such as at least 10 L, such as at least 15 L, such as at least 20 L, such as at least 25 L, such as at least 30 L, such as at least 35 L.

In some embodiments, the working volume of the scale-down reactor is at most 90% of the total volume of the scale-down reactor, wherein said working volume is defined as the amount of reaction liquid contained in the scale-down reactor.

In some embodiments the working volume of the scale-down reactor is between 50 mL and 30 L, such as between 50 mL and 600 mL, such as between 50 mL and 500 mL, such as between 50 mL and 400 mL, such as between 50 mL and 300 mL, such as between 50 mL and 200 mL, such as between 50 mL and 100 mL, such as between 100 mL and 600 mL such as between 100 mL and 500 mL, such as between 100 mL and 400 mL, such as between 100 mL and 300 mL, such as between 100 mL and 200 mL, such as between 1 L and 20 L, such as between 1 L and 10 L, such as between 1 L and 5 L.

In some embodiments, the working volume of the scale-down reactor is at least 50 mL, such as at least 60 mL, such as at least 70 mL, such as at least 80 mL, such as at least 90 mL, such as at least 100 mL, such as at least 200 mL, such as at least 300 mL, such as at least 400 mL, such as at least 500 mL, such as at least 600 mL, such as at least 700 mL, such as at least 800 mL, such as at least 900 mL, such as at least 1 L, such as at least 5 L, such as at least 10 L, such as at least 15 L, such as at least 20 L, such as at least 25 L, such as at least 30 L, such as at least 35 L.

In a particular embodiment, the working volume of the scale-down reactor can be adjusted to between 60 mL and 600 mL by modifying the number of reactor units.

In another embodiment, the working volume of the scale-down reactor can be adjusted to between 1 L and 10 L by modifying the number of reaction units.

As stated above, the scale-down reactor may comprise at least two sub-volumes, defined as the space between two adjacent disks or between one disk and the top or the bottom of the reactor. Each sub-volume may be of any volume feasible. In some embodiments, each sub-volume in the scale-down reactor has a different volume. In some embodiments, at least 2 sub-volumes have the same volume, such as at least 3 sub-volumes have the same volume, such as at least 4 sub-volumes have the same volume, such as at least 5 sub-volumes have the same volume, such as at least 6 sub-volumes have the same volume.

In some embodiments, the sub-volume is between 25 mL and 1 L, such as between 50 mL and 1 L, such as between 50 mL and 600 mL, such as between 50 mL and 500 mL, such as between 50 mL and 400 mL, such as between 50 mL and 300 mL, such as between 50 mL and 200 mL, such as between 50 mL and 100 mL, such as between 50 mL and 60 mL, such as between 75 and 150 mL, such as between 100 mL and 150 mL, such as between 400 and 700 mL, such as between 500 and 600 mL, such as between 550 and 650 mL, such as between 1 L and 1.5 L.

In some embodiments, the sub-volume is at least 50 mL, such as at least 100 mL, such as at least 200 mL, such as at least 300 mL, such as at least 400 mL, such as at least 500 mL, such as at least 600 mL, such as at least 700 mL, such as at least 800 mL, such as at least 900 mL, such as at least 1 L.

The height of the scale-down reactor is easily adjusted by modifying the number of reactor units. Thus, in some embodiments, the height of said reactor is reversibly modified by varying the number of inner reactor units.

In some embodiments, the height of the reactor is between 100 mm and 1000 mm, such as between 100 mm and 800 mm, such as between 100 mm and 600 mm, such as between 100 mm and 500 mm, such as between 100 mm and 400 mm, such as between 100 mm and 300 mm, such as between 100 mm and 200 mm, such as between 130 mm and 300 mm, such as between 350 mm and 750 mm.

In some embodiments, the height of the reactor is at least 100 mm, such as at least 120 mm, such as at least 130 mm, such as at least 140 mm, such as at least 150 mm, such as at least 160 mm, such as at least 170 mm, such as at least 180 mm, such as at least 190 mm, such as at least 200 mm, such as at least 250 mm, such as at least 300 mm, such as at least 350 mm, such as at least 400 mm, such as at least 450 mm, such as at least 500 mm, such as at least 550 mm, such as at least 600 mm, such as at least 650 mm, such as at least 700 mm, such as at least 750 mm, such as at least 800 mm, such as at least 850 mm, such as at least 900 mm, such as at least 950 mm, such as at least 1000 mm.

In a particular embodiment, the height of the scale-down reactor can be adjusted to between 130 mm and 300 mm by modifying the number of reactor units.

In another embodiment, the height of the scale-down reactor can be adjusted to between 350 mm and 750 mm by modifying the number of reaction units.

In some embodiments, the reactor subunit may be between 15 mm and 80 mm, such as between 15 mm and 25 mm, such as between 25 and 35 mm, such as between 60 and 75 mm.

In some embodiments, the reactor subunit may be between 35 and 150 mm, such as between 35 and 45 mm, such as between 100 mm and 140 mm, such as between 55 and 70 mm.

The scale-down reactor may have a particular inner diameter. In some embodiments, the inner diameter of the scale-down reactor is between 50 mm and 150 mm, such as between 60 mm and 140 mm, such as between 60 mm and 130 mm, such as between 50 mm and 100 mm, such as between 50 mm and 80 mm, such as between 50 mm and 70 mm, such as between 50 mm and 60 mm, such as between 120 mm and 150 mm, such as between 120 mm and 140 mm, such as between 13 mm and 140 mm.

In one embodiment, the inner diameter of the scale-down reactor is at least 60 mm.

In one embodiment, the inner diameter of the scale-down reactor is at least 130 mm.

The aspect ratio of the scale-down reactor is defined as the reactor height divided with the reactor inner diameter. In some embodiments, the aspect ratio of the scale-down reactor is between 1.6 and 4.2, such as 1.6, 2, 2.5, 3, 3.5, 4 or 4.2.

In one embodiment, the scale-down reactor has an aspect ratio of at least 1.5, such as at least 1.6, such as at least 1.7, such as at least 1.8, such as at least 1.9, such as at least 2, such as at least 2.1, such as at least 2.2, such as at least 2.3, such as at least 2.4, such as at least 2.5, such as at least 2.6, such as at least 2.7, such as at least 2.8, such as at least 2.9, such as at least 3, such as at least 3.1, such as at least 3.2, such as at least 3.3, such as at least 3.4, such as at least 3.5, such as at least 3.6, such as at least 3.7, such as at least 3.8, such as at least 3.9, such as at least 4, such as at least 4.1, such as at least 4.2, such as at least 4.3, such as at least 4.4, such as at least 4.5, such as at least 5.

In a particular embodiment, the aspect ratio of the scale-down reactor can be adjusted to between 1.6 and 4.2 by modifying the number of reactor units.

### Scale-Down Reactor Connections

The scale-down reactor may be assembled by connection means. In some embodiments, the connection means are reversibly and mechanically engaging elements. In some embodiments, the engaging elements are latches, fastening objects, preferably screw-like mechanisms or more preferably clicking screw-like mechanisms. In some embodiments, the engaging elements are plugs.

The connection means allow a connection between adjacent reactor units or between a reactor unit and the top unit or between a reactor unit and the bottom unit, such that liquid contained within one unit can pass to the adjacent unit, and such that entire scale-down reactor is separated in a fluid-tight manner from the outer environment, as will be readily understood by the skilled person. Thus any connection means allowing two reactor units (i.e. between two adjacent inner reactor units, or between an inner reactor unit and the top unit, or between an inner reactor unit and the bottom unit) to be connected in such a manner while maintaining controlled interactions with the outer environment (e.g. sterile conditions) can be employed. Such connection means are readily available to the skilled person.

In preferred embodiments, the connection means are screw-like mechanisms. In such embodiments, the units are connected by screwing them onto one another and clicking them tight.

In some embodiments, the connection means between any two adjacent reactor units (i.e. between two adjacent inner reactor units, or between an inner reactor unit and the top unit, or between an inner reactor unit and the bottom unit) are all the same. In other embodiments, the connection means between any two adjacent reactor units are different.

### Scale-Down Reactor Features

The scale-down reactor may comprise one or more features, such as inlet and/or outlet apertures and/or apertures for observation of the inside of the reactor, such as for observation of the reaction liquid.

Thus, in some embodiments, one or more reactor units of the scale-down reactor comprises an observation aperture. In preferred embodiments, the top unit comprises an observation aperture. The observation aperture can be useful for the user to visually monitor the reaction; for example to visually detect foam formation.

In some embodiments, one or more reactor units of the scale-down reactor comprises one or more inlet apertures. In preferred embodiments, said inlet apertures allow for the injection and/or sampling of liquids, solids or gases, preferably in a fluid-tight manner.

In some embodiments, one or more reactor units of the scale-down reactor comprises one or more outlet apertures. In preferred embodiments, said outlet apertures allow for the injection and/or sampling of liquids, solids or gases, preferably in a fluid-tight manner.

Such inlet and outlet apertures may be as are known in the art, and are such that contamination of the liquid within the scale-down reactor can be avoided.

One or more inlet apertures may for example allow for the injection of a pH-adjusting compound such as base and/or acid; substrate(s) required for the bio-based production process; anti-foam for foam control; gases, such as air; and inoculum, i.e., cells/microorganisms.

An inlet aperture in a specific reactor unit, such as in a sub-volume of the scale-down reactor, may allow for the injection of a compound, such as a substrate, in said specific sub-volume in order to evaluate the mixing of the compound injected in said sub-volume.

One or more outlet apertures may for example allow for sampling of the reaction liquid, such as sampling for evaluating microbial or cell growth, dissolved oxygen, pH, real-time OD measurement, real-time flow cytometry, and concentration of one or more metabolites, such as concentration of one or more product(s) and one or more substrate(s).

An outlet aperture in a specific reactor unit, such as in a sub-volume of the scale-down reactor, may allow for sampling in said specific sub-volume in order to measure microbial or cell growth, and concentration of one or more metabolites, such as concentration of one or more product(s), one or more inhibitor(s) and/or one or more substrate(s) in the reaction liquid of said specific sub-volume.

Thus, in some embodiments, at least one reactor unit comprises one or more inlet and/or outlet apertures, such as wherein at least 2 reactor units comprises one or more inlet and/or outlet apertures, such as wherein at least 3 reactor units comprises one or more inlet and/or outlet apertures, such as wherein at least 4 reactor units comprises one or more inlet and/or outlet apertures, such as wherein at least 5 reactor units comprises one or more inlet and/or outlet apertures, such as wherein at least 6 reactor units comprises one or more inlet and/or outlet apertures, such as wherein at least 7 reactor units comprises one or more inlet and/or outlet apertures, such as wherein all reactor units comprises one or more inlet and/or outlet apertures, allowing for the injection and/or sampling of liquids, solids or gases, preferably in a fluid-tight manner.

In some embodiments, the scale-down reactor comprises one or more inner reactor unit(s). In some embodiments, at least one of said inner reactor units comprises one or more inlet and/or outlet apertures, such as wherein all of said one or more inner reactor unit(s) comprises one or more inlet and/or outlet apertures.

One or more apertures of the scale-down reactor may further allow for the insertion of a pH probe, a dO (dissolved oxygen) probe, a temperature probe, a real-time OD probe, a biomass probe, a sparger and/or a mixer, i.e., a stirrer. In preferred embodiments, apertures for insertion of a pH probe, a dO probe, a temperature probe, a sparger and/or a mixer, i.e., a stirrer, are located on top of the top unit of the scale-down reactor.

Thus, in some embodiments, the top unit comprises at least 4 inlet and/or outlet apertures, such as at least 5 inlet and/or outlet apertures, such as at least 6 inlet and/or outlet apertures, such as at least 7 inlet and/or outlet apertures.

One or more inlet aperture of the scale-down reactor may allow for the injection of gases, such as air, into the reaction liquid. In some embodiments, an aperture at the top unit of the scale-down reactor allows for the insertion of a sparger that can inject gases, such as air, into the reaction liquid. However, in preferred embodiments, the bottom unit comprises inlet apertures for injection of gases into the reaction liquid, such as inlet apertures for the injection of air, i.e. inlet apertures for aerating the reaction liquid.

The scale-down reactor may further comprise baffles. Baffles are flow-directing or obstructing vanes or panels used to direct a flow of liquid or gas. Baffles may improve the mixing time of a reaction liquid. The baffles may have any shape suitable as is known in the art.

Preferably, the baffles are placed on the inner wall of the reactor, such as on the inner wall on the one or more reactor unit(s), preferably on the inner wall of one or more of the inner reactor unit(s).

Thus, in some embodiments, the scale-down reactor comprises at least one baffle, such as at least two baffles, such as at least 3 baffles, such as at least 4 baffles, such as at least 5 baffles, such as at least 6 baffles, such as at least 7 baffles, such as at least 8 baffles, such as at least 9 baffles, such as at least 10 baffles. In some embodiments, each inner reactor unit comprises at least one baffle, such as two baffles.

However, in other embodiments, the scale-down reactor does not comprise any baffles.

### Temperature Control Jacket

The scale-down reactor may be enclosed by a device that is configured to regulate, i.e. modify, the temperature of the reaction liquid comprise within the scale-down reactor.

Thus, in some embodiments, the scale-down reactor is enclosed by a temperature control jacket (10) comprising one or more jacket units (11, 12, 13), said control jacket being configured for modifying the temperature of the reaction fluid contained in the reactor.

In some embodiments, the temperature control jacket comprises a top unit (11) and a bottom unit (12) and optionally one or more middle jacket units (13). In some embodiments, the total jacket height is reversibly modified by varying the number of jacket units, in particular the number of middle jacket units. In some embodiments, the jacket encloses at least the bottom unit and the optional one or more further inner reactor units of the scale-down reactor.

In some embodiments, the jacket units are reversibly attached to one another via connection means. Such connection means may for example be screws and/or plugs and/or connection means described herein in the section “Scale-down reactor connections”.

The temperature control jacket may regulate the temperature of the reaction liquid by a flow, through the jacket, of a heat transfer liquid, such as water. Thus, in some embodiments, the temperature control jacket comprises one inlet aperture (14) and one outlet aperture (15) to allow for a heat transfer liquid such as water to flow through the jacket.

### Mixing—Stirrer and Impeller

The scale-down reactor may for example be operated as a stirred tank reactor or as a bubble column reactor. In some embodiments, the scale-down tank reactor is operated as a stirred tank reactor. Thus, in some embodiments, the scale-down reactor comprises a stirrer, i.e. a mixing shaft wherein said mixing shaft comprises at least one impeller.

The mixing shaft may be modular. Thus, in some embodiments, the total length of the mixing shaft is reversibly modified by varying the number of mixing shaft units.

In some embodiments, the mixing shaft comprises an impeller and is configured such that the impeller, once the scale-down reactor is assembled, is located in at least one sub-volume, such as in at least 2 sub-volumes, such as in at least 3 sub-volumes, such as in at least 4 sub-volumes, such as in at least 5 sub-volumes, such as in at least 6 sub-volumes, such as in at least 7 sub-volumes, such as in each sub-volume of the scale-down reactor.

There are no restrictions as to which type of impeller can be used in the scale-down reactor. In other words, any type of impeller providing the desired mixing conditions can be used. In some embodiments, the impeller is selected from the group consisting of an axial flow impeller, a tangential flow impeller, a radial flow impeller, a high shear impeller and a specialty impeller or combinations thereof. In some embodiments, the impeller is selected from the group consisting of Rushton turbine, cross beam, frame impeller, anchor impeller, flat-blade impeller, pointe blade impeller, propeller, pitched blade turbine, hydrofoils, hollow-blade turbine, helical ribbon, cowls, disk, bar, pointed blade impeller, retreat curve impeller, sweptback impeller, spring impeller and glass-lined turbine. In specific embodiments, the impeller is a Rushton turbine.

### Disks

The scale-down reactor comprises at least one disk. In some embodiments, said disk is reversibly inserted transversally or perpendicularly to the main axis of the reactor unit. In some embodiments, the space between two adjacent disks or between one disk and the top or the bottom of the reactor defines a sub-volume from the total volume of the reactor. In some embodiments, the at least one disk separates the sub-volumes created by adjacent reactor units, such as between two inner reactor units, or between the top unit and the adjacent reactor unit, or between the bottom unit and the adjacent reactor unit. However, the scale-down reactor can comprise adjacent reactor units which are not separated by a disk. For instance, a scale-down reactor comprises a top unit, a bottom unit and two inner units, and comprises one single disk between two units, e.g. between the two inner units; or the scale-down reactor comprises a disk separating the bottom unit and the adjacent inner unit, and a disk separating the top unit and the adjacent inner unit, but no disk is placed between the two adjacent inner units.

If the scale-down reactor comprises n inner reactor units, where n is an integer, then the scale-down reactor preferably comprises at the most n+1 disks.

The disk has approximately the same diameter as the inner diameter of the scale-down reactor. Thus, in some embodiments, the disk has a diameter of approximately between 50 mm and 150 mm, such as between 60 mm and 140 mm, such as between 60 mm and 130 mm, such as between 50 mm and 100 mm, such as between 50 mm and 80 mm, such as between 50 mm and 70 mm, such as between 50 mm and 60 mm, such as between 120 mm and 150 mm, such as between 120 mm and 140 mm, such as approximately between 13 mm and 140 mm.

In one embodiment, the disk has a diameter of approximately at least 60 mm.

In one embodiment, the disk has a diameter of approximately at least 130 mm.

In some embodiments, the scale-down reactor comprises at least 2 disks, such as at least 3 disks, such as at least 4 disks, such as at least 5 disks, such as at least 6 disks, such as at least 7 disks, such as at least 8 disks.

The disk comprises at least one aperture, allowing the flow of liquid between adjacent sub-volumes, such as between adjacent reactor units. In some embodiments, the disk comprises a shaft aperture, allowing a mixing shaft as well as fluid contained in adjacent sub-volumes to pass through it.

In some embodiments, the disk comprises at least 1 aperture, such as at least 2 apertures, such as at least 3 apertures, such as at least 4 apertures, such as at least 5 apertures, such as at least 6, such as at least 7 apertures, such as at least 8 apertures, such as at least 9 apertures, such as at least 10 apertures, such as at least 11, such as at least 12 apertures, such as at least 13 apertures, such as at least 14 apertures, such as at least 15 apertures, such as at least 16, such as at least 17 apertures, such as at least 18 apertures, such as at least 19 apertures, such as at least 20 apertures, such as at least 25, such as at least 30 apertures, such as at least 35 apertures, such as at least 40 apertures, such as at least 45 apertures, such as at least 50 apertures, such as at least 60 apertures, such as at least 70 apertures, such as at least 80 apertures, such as at least 90 apertures, such as at least 100 apertures, such as at least 200 apertures, such as at least 300 apertures, such as at least 400 apertures, such as at least 500 apertures, such as at least 600 apertures, such as at least 700 apertures, such as at least 800 apertures, such as at least 900 apertures, such as at least 1000 apertures, such as at least 1100 apertures, such as at least 1200 apertures, such as at least 1300 apertures, such as at least 1400 apertures, such as at least 1500 apertures.

In some embodiments, the disk comprises between 1 and 1500 apertures, such as between 1 and 1000 apertures, such as between 5 and 1000 apertures, such as between 10 and 1000 apertures, such as between 100 and 1000 apertures, such as between 200 and 1000 apertures, such as between 500 and 1000 apertures, such as between 600 and 1000 apertures, such as between 700 and 1000 apertures, such as between 800 and 1000 apertures, such as between 900 and 1000 apertures, such as between 1 and 500 apertures, such as between 5 and 500 apertures, such as between 10 and 500 apertures, such as between 50 and 500 apertures, such as between 100 and 500 apertures, such as between 200 and 500 apertures, such as between 300 and 500 apertures, such as between 400 and 500 apertures, such as between 1 and 100 apertures, such as between 5 and 100 apertures, such as between 10 and 100 apertures, such as between 50 and 100 apertures, such as between 1 and 50 apertures, such as between 5 and 50 apertures, such as between 10 and 50 apertures, such as between 20 and 50 apertures, such as between 30 and 50 apertures, such as between 40 and 50 apertures, such as between 1 and 20 apertures, such as between 1 and 10 apertures, such as between 1 and 5 apertures, such as between 20 and 200 apertures, such as between 200 and 800 apertures, such as between 2 and 1000 apertures, such as between 3 and 300 apertures.

There are no restrictions as to the shape of the aperture(s) of the disk. In other words, the aperture(s) may be of any shape. However, the aperture(s) should preferably be easy to clean. Thus, in preferred embodiments, the aperture(s) are circular.

There are no restrictions as to the size of the aperture(s). However, an aperture may of course not be larger than the disk.

In some embodiments, the apertures are circular.

In some embodiments, the apertures are between 0.5 mm and 100 mm in diameter, such as between 50 mm and 100 mm in diameter, such as between 0.5 mm and 50 mm in diameter, such as between 0.5 mm and 40 mm in diameter, such as between 0.5 mm and 30 mm in diameter, such as between 0.5 mm and 20 mm in diameter, such as between 0.5 mm and 10 mm in diameter, such as between 0.5 mm and 5 mm in diameter, such as between 0.5 mm and 4 mm in diameter, such as between 0.5 mm and 3 mm in diameter, such as between 0.5 mm and 2 mm in diameter, such as between 0.5 mm and 1 mm in diameter, such as between 1 mm and 2 mm in diameter, such as between 2 mm and 100 mm in diameter, such as between 2 mm and 50 mm in diameter, such as between 2 mm and 40 mm in diameter, such as between 2 mm and 30 mm in diameter, such as between 2 mm and 20 mm in diameter, such as between 2 mm and 10 mm in diameter.

In some embodiments, the apertures are at least 0.5 mm in diameter, such as at least 1 mm, such as at least 2 mm, such as at least 3 mm, such as at least 4 mm, such as at least 5 mm, such as at least 6 mm, such as at least 7 mm, such as at least 8 mm, such as at least 9 mm, such as at least 10 mm, such as at least 15 mm, such as at least 20 mm, such as at least 25 mm, such as at least 50 mm, such as at least 75 mm, such as at least 100 mm in diameter.

The aperture(s) allow exchange of reaction fluid between adjacent sub-volumes of the scale-down reactor. The area of the aperture(s) is thus one factor determining the amount of reaction fluid that is exchanged between adjacent sub-volumes. Thus, the total area of the aperture(s) may be termed the specific exchange area. Thus, in some embodiments, the size, shape and number of apertures is selected to provide a specific exchange area between the sub-volumes of the scale-down reactor.

Further, the aperture(s) allow reaction fluid to flow between adjacent sub-volumes of the scale-down reactor, generating a specific flow profile between said sub-volumes. The specific flow profile is determined by the specific exchange area and, if present, by the type and speed of the impeller. Thus, in some embodiments, the size, shape, number and location of the apertures, and/or the impeller and impeller speed, is selected to provide a specific flow profile between adjacent sub-volumes, such as between sub-volumes of adjacent reactor units.

In some embodiments, the specific exchange area and/or the specific flow profile between adjacent sub-volumes, such as between sub-volumes of adjacent reactor units, are designed to provide a specific mixing time in each sub-volume, such as in each sub-volume of each adjacent reactor unit. In preferred embodiments, the mixing time is different in each sub-volume, such as in each sub-volume of adjacent reactor units.

Preferably, the mixing time of a sub-volume of a reactor unit is defined as the time required for achieving a pre-determined degree of homogeneity in said sub-volume of said reactor unit.

In some embodiments, the mixing time of a sub-volume is dependent on the specific exchange area and/or the specific flow profile between adjacent sub-volumes; the fluid density; and the type and speed of the impeller. Thus, in some embodiments, the mixing time of a sub-volume is dependent on the specific exchange area and/or the specific flow profile and on the Power Input Value as described in the section “Method of simulating large-scale reactor”.

In preferred embodiments, the one or more disk with one or more aperture is selected so that the mixing time of the scale-down reactor mimics the mixing time of a large-scale or pilot scale reactor, as described in the section “Method of simulating large-scale reactor”. In other words, the one more disk with one or more aperture may be selected so that the conditions and/or the operating conditions of the scale-down reactor simulate and/or mimic the conditions and/or the operating conditions of a large-scale or pilot scale reactor, such that sub-volumes with varying mixing times of the scale-down reactor simulate and/or mimic zones with varying mixing times of the large-scale or pilot scale reactor.

The disk and the reactor unit in which the disk is to be placed are preferably configured such that once in place, the disk is maintained in place even in operating conditions. The disk can thus be connected to the reactor unit (inner reactor unit, top unit or bottom unit) for example via the presence of a recess on the inner diameter of the reactor unit, or via hinges or latches. It will be understood that the disk can be placed at any position (with reference to a vertical axis) within the reactor unit.

### Set-Up of the Scale-Down Reactor

The set-up of the scale-down reactor refers to the physical design of the reactor, such as for example the number of reactor units, the number of disks, the number of apertures (i.e., the disk design), the (if any) type and number of impeller(s). The set-up of the scale-down reactor to use may for example be selected as described in this section. The skilled person will however have no difficulty in designing the set-up of the scale-down reactor.

The scale-down reactor may be operated in a defined scale-down mode depending upon the operating conditions of a large-scale reactor process. Large-scale reactors may for example be operated in a defined mode, such as bubble column or stirred tank reactor. The reactor may have a specific aspect ratio and, if it is a stirred tank reactor, a specific number of impeller.

With an understanding of large-scale conditions, the relevant conditions may be achieved by mimicking a similar liquid phase mixing time or liquid flow distributions of a large-scale reactor in the scale-down reactor. Rational assessment of large-scale reactor conditions may be obtained from Computational Fluid Dynamic (CFD) simulations, for example using software program like ANSYS-Fluent, CFX, OpenFOAM, or similar. Assessment may also be done by a compartmental modelling approach. The conditions may be mimicked in the scale-down reactor using CFD or by determining the mixing time in sub-volumes with various disk designs experimentally. In other words, the choice of the disk design may depend on the mixing time or the liquid flow distribution that needs to be represented in the scale-down reactor at a certain volume. In some instances, a higher number of apertures in the disk, may give a higher liquid flow in axial distribution.

In some embodiments, the set-up of the scale-down reactor is chosen based on the operational mode of the pilot-scale or large-scale reactor to be mimicked, modified, modelled or monitored. In other words, the set-up of the scale-down reactor is the same or similar as to the pilot-scale or large-scale reactor in terms of aspect ratio, operational mode and/or mixing ability (such as impeller design and mixing time).

In some preferred embodiments, the set-up of the scale-down, such as the number of disks and the design of said disks, is determined based on the mixing time.

Thus, in preferred embodiments, the scale-down reactor is set-up so that the mixing time of said scale-down reactor is the same or similar to the mixing time of the pilot scale or large-scale reactor which conditions are to be simulated, modelled, mimicked, monitored or modified.

In some embodiments, the mixing time of one or more set-ups of the scale-down reactor may be modelled using computational software. In some embodiments, one or more conditions or parameters of the scale-down reactor, such as temperature, pH, gas concentration, metabolite concentration, cellular mass, cellular density, enzymatic kinetics, absorbance, and/or mixing time, may be modelled using computational software. Said conditions or parameters may be modelled by providing a computing unit containing a digital representation of the scale-down reactor, receiving data from the at least one probe, and varying operating conditions of the scale-down reactor until a simulated optimal state provided by the digital representation of the scale-down reactor is achieved.

In some embodiments, the simulated optimal state provided by the digital representation may be understood as a simulated state that fulfil optimal growth, fermentation, incubation and/or required process conditions for a specific reaction being performed in the scale-down reactor.

The digital representation of the scale-down reactor is indistinguishable from the scale-down reactor, and may be a digital twin model. Examples of digital representation of the scale-down reactor include, but are not limited to, modelling system such as a digital twin model, a machine learning model or a neural network model. A digital twin may be a virtual model designed to indistinguishably reflect the scale-down reactor. The sensors located in areas of the scale-down reactor measuring conditions such as temperature, pH, gas concentration, metabolite concentration, cellular mass, cellular density, enzymatic kinetics, absorbance, and/or mixing time produce data about the physical scale-down reactor. The produced data is then input to a processing unit and applied to the digital copy. For the purpose of modelling the scale-down reactor and predicting a future state of the conditions being measured on reactor, the digital twin is indistinguishable from the scale-down reactor.

The digital representation of the scale down reactor may be used for varying operating conditions of the scale-down reactor to reach a simulated optimal state by the digital representation of the scale-down reactor. Said simulated optimal state may be used to analyse the performance of the scale-down reactor issues and generate optimized conditions on the reactor for a specific experiment. The acquisition of data from the scale-down reactor and the implementation of the optimal conditions calculated by the digital representation may be performed in real-time, achieving a real-time optimal control of the scale-down reactor, for a specific experiment.

Said optimal state may be achieved by varying in real-time the conditions that control the scale-down reactor, such as temperature, pH, gas concentration, metabolite concentration, cellular mass, cellular density, enzymatic kinetics, absorbance, and/or mixing time.

In other embodiments, the mixing time of one more set-ups of the scale-down reactor may be determined experimentally, such as by measuring the mixing time in various set-ups of the scale-down reactor, such as by using the method as described herein in the section “Examples/Suitability of the MSTR for scale-down operation”.

### Method of Simulating Large-Scale Reactor

The scale-down reactor of the present invention is especially suitable for modelling and/or mimicking the conditions of a large-scale or pilot scale reactor, such as for modelling and/or mimicking gradients that are likely to occur at larger scale. The conditions include reaction reaction volume, such as reaction liquid volume and/or reaction gas volume, temperature, pH, gas concentration, metabolite concentration, cellular mass, cellulose density, cellular concentration (such as optical density (OD) of cells), enzymatic kinetics, absorbance and mixing time.

Heterogenic conditions, i.e. gradients, may for example arise due to mixing challenges present at larger scale. In particular, the scale-down reactor allows for identification of such mixing challenges prior to, during or after scaling up a reactor production process.

The present invention thus provides a method of simulating, modelling, monitoring or modifying the conditions of a reactor comprising a liquid composition in a bioreactor or live fermentation process, comprising the steps of:
a. providing the scale-down reactor as described herein;
b. defining the total reactor volume by reversibly adding a number of inner reactor units (3);
c. defining the number, position and design of the at least one disk (7); and
d. optionally, defining the design of the mixing shaft (5) and one or more impeller (6);
wherein the conditions to be simulated, modelled, monitored or modified are selected from the group consisting of: reaction volume, temperature, pH, gas concentration, metabolite concentration, cellular mass, cellular density, enzymatic kinetics, absorbance, and mixing time, and
wherein the reactor is at least 300 L.

In some embodiments, the present invention provides a method of mimicking the conditions of a reactor comprising a liquid composition in a bioreactor or live fermentation process, comprising the steps of:
a. providing the scale-down reactor according as described herein;
b. defining the total reactor volume by reversibly adding a number of inner reactor units (3);
c. defining the number, position and design of the at least one disk (7); and
d. optionally, defining the design of the mixing shaft (5) and one or more impeller (6);
wherein the conditions to be mimicked are selected from the group consisting of: reaction volume, temperature, pH, gas concentration, metabolite concentration, cellular mass, cellular density, enzymatic kinetics, absorbance, and mixing time, and wherein the reactor is at least 300 L.

The reaction compartment is fluid-tight and can received, or is configured to receive, a reaction liquid, a reaction gas, or a combination of reaction liquid and reaction gas. Thus in some embodiments, the reaction compartment comprises a reaction liquid, and optionally a reaction gas, such as a gaseous substrate.

In some embodiments, said one or more conditions is simulated, modelled, monitored or modified independently in each sub-volume of the scale-down reactor.

The reactor as referred to herein is the larger scale reactor, the conditions of which the user wants to simulate, monitor, modify, mimic or model, and is preferably a pilot scale or large-scale reactor. In principle, the reactor can be of any size that allows for gradients, i.e. heterogeneity of the reaction liquid, to occur. Preferably, said reactor is at least 300 L, such as at least 400 L, such as at least 500 L, such as at least 600 L, such as at least 700 L, such as at least 800 L, such as at least 900 L, such as at 1000 L, such as at least 1500 L, such as at least 2000 L, such as at least 3000 L, such as at 4000 L, such as at least 5000 L, such as at least 6000 L, such as at least 7000 L, such as at 8000 L, such as at least 9000 L, such as at least 10,000 L, such as at least 25,000 L, such as at 50,000 L, such as at least 75,000 L, such as at least 100,000 L, such as at least 500,000 L.

The scale-down reactor is preferably the same type of reactor as the reactor. Further, the scale-down reactor is preferably operated in the same mode as the reactor.

Thus, in some embodiments, the scale-down reactor is operated in the same mode as the reactor, such as in batch, fed-batch or continuous mode. In some embodiments, the scale-down reactor is of the same type as the reactor, wherein the type is selected from a stirred tank reactor and a bubble column reactor.

Preferably, the scale-down reactor is operated under similar operating conditions as the large-scale or pilot scale reactor. In particular, the scale-down reactor may be operated with the same mixing time, Power input value (P/V), impeller tip speed, volumetric mass transfer coefficient and/or oxygen transfer rate as that of the large-scale or pilot scale reactor. Further preferably, the scale-down reactor has the same aspect ratio as the large-scale or pilot scale reactor.

Thus, in some embodiments one or more operating conditions in the scale-down reactor are equal or similar to said corresponding operating conditions in the reactor, wherein said operating conditions are selected from:
Power input value (P/V);
aspect ratio;
impeller tip speed;
mixing time;
volumetric mass transfer coefficient; and/or
oxygen transfer rate.

In some embodiments, the Power Input Value (P/V) of the scale-down reactor is equal or similar to the P/V of the reactor, wherein P is the Power Input and V is the Volume in the reactor, and wherein P may be calculated according to the formula:

$$P = Po ⋆ ρ ⋆ N ⋆ {D}^{3}$$

wherein V is the volume of the reaction liquid, Po is the Power number for the impeller, ρ is the fluid density, N is the rotational speed of the impeller and D is the impeller diameter.

In some embodiments, the volumetric mass transfer coefficient (k_{L}a) and/or the oxygen transfer rate (OTR) of the scale-down reactor is equal or similar to the k_{L}a and/or the OTR of the reactor, wherein said coefficient and rate may be calculated according to the formula:

$${k}_{L} a = OTR / ( {C}_{L}^{⋆} - {C}_{L} )$$

wherein C*_{L} is the dissolved oxygen concentration at air saturation and C_{L} is the dissolved oxygen concentration in the liquid phase.

In some embodiments, the mixing time of the scale-down reactor is equal or similar to the mixing time of the reactor, wherein said mixing time is defined as the time required for achieving a pre-determined degree of homogeneity in the reactor.

In some embodiments, the scale-down reactor and the reactor is a bioreactor, such as a live fermentation process. Thus, in some embodiments, the method disclosed herein comprises cultivation of cells, such as eukaryotic or prokaryotic cells, such as Chinese Hamster Ovary (CHO) cells, yeast, fungi and/or bacteria.

### Use of the Reactor

The scale-down reactor of the present invention can be used to simulate, model, mimic, monitor and/or modify the conditions of a reactor, preferably a pilot scale or large-scale reactor.

Thus, the present invention provides for the use of a scale-down reactor as described herein for simulating, modelling, monitoring or modifying the conditions of a reactor, wherein the conditions to be simulated, modelled, monitored or modified are selected from the group consisting of: reaction volume, temperature, pH, gas concentration, metabolite concentration, cellular mass, cellular density, enzymatic kinetics, absorbance, and mixing time, wherein the reactor is at least 300 L.

In some embodiments, said one or more conditions is simulated, modelled, monitored or modified in each sub-volume of the scale-down reactor.

Preferably, the scale-down reactor for use is operated under the same operating conditions as the reactor, such as described herein above in the section “Method for simulating large-scale reactor. Further preferably, the scale-down reactor is operated in the same mode and is the same type as the reactor, as also described herein above in the section “Method for simulating large-scale reactor”. The reaction compartment is fluid-tight and can received, or is configured to receive, a reaction liquid, a reaction gas, or a combination of reaction liquid and reaction gas. Thus in some embodiments, the reaction compartment comprises a reaction liquid and optionally a reaction gas, such as a gaseous substrate.

The reactor as referred to herein is preferably a pilot scale or large-scale reactor as described herein in the section “Method for simulating large-scale reactor”.

In some embodiments, the scale-down reactor for use is a bioreactor or a live fermentation process. Thus, in some embodiments, the scale-down reactor for use comprises the cultivation of cells, such as eukaryotic or prokaryotic cells, such as Chinese Hamster Ovary (CHO) cells, yeast, fungi or bacteria.

However, in some embodiments, the present invention provides the use of a scale-down reactor as described herein for liquid-liquid extraction.

### Adaptive Laboratory Evolution

It is well known that microorganisms can adapt to various conditions under selection pressure. Adaptive laboratory evolution (ALE) is a frequent method in biological studies to engineer microbial cells for biotechnological applications. During microbial ALE, a microorganism is cultivated under defined conditions for prolonged periods of time, which allows the selection of improved phenotypes (Dragotis and Mattanovich, 2013). Such phenotypes include for example improved growth, improved substrate utilization, increased tolerance to inhibitors/toxic compounds, and increased tolerance to the surrounding conditions/environment in general.

ALE experiments can for example be conducted in batch, repeated batch, serial batch, fed-batch and/or continuous (i.e. chemostat) conditions, usually during weeks or months in order to allow for a certain number of generations or cumulative number of cell divisions (CCD) to occur (Dragotis and Mattanovich, 2013; Lee et al., 2011).

CCDs can be calculated as follows (Lee et al., 2011):

$$CCD = {\sum }_{i = 1}^{m} {N}_{0 , i} ( {2}^{ni} - 1 )$$

Where, N_{0,i} is the initial number of cells in each flask during the evolution, n is the number of generations for each flask, and m is the total number of individual flask cultures used in the serial ALE process.

Number of generations can be calculated as follows (Lee et al., 2011):

$$n = \frac{log ( N / {N}_{0} )}{log 2}$$

Where, N is the final number of cells in a flask at the time of passage to the next flask

As stated above in the section “Background”, scale-down reactors can be used for ALE, i.e. to evolve strains that are more robust and better tolerate a large-scale environment.

Thus, further provided herein is a method of adapting a microorganism to the conditions of a reactor, said method comprising:
a. providing the scale-down reactor according to the present invention;
b. defining the total reactor volume by reversibly adding a number of inner reactor units (3);
c. defining the number, position and design of the at least one disk (7);
d. optionally, defining the design of the mixing shaft (5) and one or more impellers (6); and
e. growing the microorganism in the scale-down reactor for an extended amount of time in order to allow the microorganism to adapt to the conditions of the reactor;
wherein the reactor is at least 300 L,
thereby obtaining an adapted microorganism.

The reactor referred to is preferably a pilot scale or large-scale reactor as described herein in the section “Method for simulating large-scale reactor”. Thus, in some embodiments, the conditions are selected from the group consisting of: fluctuating temperature, fluctuating pH, fluctuating metabolite concentration, fluctuating gas concentration, and fluctuating oxygen concentration. In other words, the adapted microorganism is more robust and tolerable to said conditions being fluctuating.

Preferably, the method of adapting a microorganism to the conditions of a reactor results in a microorganism with improved growth in a large-scale or pilot scale reactor, such as improved growth compared to a similar (e.g. of the same strain) microorganism not having been adapted according to the method disclosed herein, when said microorganisms are grown under the same conditions. Microbial growth can be measured using techniques well known in the art. For example, microbial growth can be measured by measuring the absorbance of the reaction liquid at 600 nm (i.e. by measuring the optical density (OD) at regular intervals.

Thus, in some embodiments, the adapted microorganism has acquired adaptive mutations that improves the growth of said microorganism as compared to another microorganism of the same type, e.g. the same strain, which has not acquired said adaptive mutations, when said microorganisms are grown under the same conditions in a reactor.

In some embodiments, the adapted microorganism has a higher growth rate as compared to another microorganism of the same type, e.g. the same strain, which has not acquired said adaptive mutations, when said microorganisms are grown under the same conditions in a reactor.

In some embodiments, the adapted microorganism has acquired adaptive mutations that improves the production of said microorganism of a certain metabolite as compared to another microorganism of the same type, e.g. the same strain, which has not acquired said adaptive mutations, when said microorganisms are grown under the same conditions in a reactor.

The present invention further provides for the use of a scale-down reactor as described herein for adapting a microorganism to the conditions of a reactor.

The microorganism may have acquired any appropriate number of adaptive mutations required for said microorganism being able to adapt to the conditions of the reactor.

In some embodiments, adapting the microorganism to the conditions of a reactor comprises the microorganism acquiring one or more adaptive mutations. In some embodiments, the microorganism acquires at least one adaptive mutation, such as at least 2 adaptive mutations, such as at least 3 adaptive mutations, such as at least 4 adaptive mutations, such as at least 5 adaptive mutations, such as at least 6 adaptive mutations, such as at least 7 adaptive mutations, such as at least 8 adaptive mutations, such as at least 9 adaptive mutations, such as at least 10 adaptive mutations.

In some embodiments, the microorganism acquires between 2 and 10 adaptive mutations, such as between 2 and 8 adaptive mutations.

The microorganism may be grown in the reactor for any amount of time appropriate for allowing said microorganism to adapt to the conditions of the reactor.

In some embodiments, the microorganism is grown for at least one week in the scale-down reactor, such as at least 2 weeks, such as at least 3 weeks, such as at least 4 weeks, such as at least 5 weeks, such as at least 6 weeks, such as at least 7 weeks, such as at least 8 weeks, such as at least 2 months, such as at least 3 months, such as at least 4 months, such as at least 5 months, such as at least 6 months, such as at least 7 months, such as at least for 1 year in the scale-down reactor.

In some embodiments, the microorganism is grown for between 2 weeks and 1 year in the scale-down reactor, such as between 4 weeks and 8 weeks, such as between 1 month and 6 months.

In some embodiments, the microorganism is grown in the scale-down reactor for at least 10⁹ CCDs, such as at least 10¹⁰ CCDs, such as at least 10¹¹ CCDs, such as at least 10¹² CCDs, such as at least 10¹³ CCDs, such as at least 10¹⁴ CCDs, such as at least 10¹⁵ CCDs, such as at least 10²⁰ CCDs in the scale-down reactor.

In some embodiments, the microorganism is grown in the scale-down reactor for between 10⁹ CCDs and 10²⁰ CCDs, such as between 10¹⁰ CCDs and 10¹³ CCDs, such as between 10¹¹ CCDs and 10¹² CCDs.

In some embodiments, the microorganism is grown in the scale-down reactor for at least 150 generations, such as at least 200 generations, such as at least 250 generations, such as at least 300 generations, such as at least 350 generations, such as at least 400 generations, such as at least 450 generations, such as at least 500 generations, such as at least 600 generations, such as at least 700 generations, such as at least 800 generations, such as at least 900 generations, such as at least 1000 generations, such as at least 1500 generations, such as at least 2000 generations, such as at least 3000 generations, such as at least 4000 generations, such as at least 5000 generations, such as at least 10,000 generations.

In some embodiments, the microorganism is grown in the scale-down reactor for between 150 and 10,000 generations, such as between 200 and 5000 generations, such as between 300 and 2000 generations, such as between 500 and 2000 generations, such as between 200 and 1000 generations.

### System and Manufacture

The present invention provides a system comprising the scale-down reactor as described herein, and a reaction fluid, such as a reaction liquid and/or a reaction gas. Thus in some embodiments, the reaction compartment comprises a reaction liquid and optionally a reaction gas, such as a gaseous substrate.

The present invention further provides a method of manufacturing a scale-down reactor, said method comprising:
a. providing
   i. a reaction compartment comprising at least two reactor units, wherein said at least two reactor units are reversibly attached to one another via connection means,
   ii. wherein the at least two reactor units comprise a top unit (1) and a bottom unit (2), and optionally comprise one or more further inner reactor units (3); wherein said reactor units when attached to one another form a fluid-tight reaction compartment for receiving or configured to receive a reaction liquid therein; wherein one or more of said units comprise one or more inlet fluid apertures (4) and one or more outlet fluid apertures (4),
   iii. optionally, a mixing shaft (5) placed or configured to be placed vertically in the reaction compartment, said mixing shaft comprising one or more impellers (6), and
   iv. at least one disk (7) inserted or configured to be inserted horizontally between two adjacent reactor units or horizontally within an inner reaction unit, said disk comprising at least one aperture (16) allowing the fluid contained in the adjacent reactor units to pass through it, optionally said disk comprising at least one shaft aperture (8) allowing the mixing shaft to pass through it;
b. assembling said reactions units, disk and optionally said mixing shaft.

The individual elements of the scale-down reactor are preferably as described herein above.

### EXAMPLES

Microbial Cultivation with the Scale-Down Reactor

### Materials and Methods

### Cultivation Medium and its Preparation

The composition of the pre-culture and the batch medium used for cultivating the strain in shake flask and in the scale-down reactor are mentioned in the table below. The scale-down reactor was run as a stirred tank reactor and is referred to as a modular stirred tank reactor, i.e., MSTR, herein below and in the figure and figure descriptions.

TABLE 1
Composition of the pre-culture medium used to grow overnight precultures
in shake flask.

| Component | Target amount | Units |
|---|---|---|
| Glucose stock 20% | 10 | mL/L |
| M9 salt stock solution | 100 | mL/L |
| Magnesium sulfate (2M) | 1 | mL/L |
| (MgSO₄) | | |
| M9 trace element stock | 0.5 | mL/L |
| solution | | |
| M9 vitamin stock solution | 1 | mL/L |
| Calcium dichloride stock | 1 | mL/L |
| (1M) (CaCl₂) | | |
| Water | Fill to 1L total volume | mL/L |

TABLE 2
Composition of the batch medium used for both reference and scale-down
setups. Ammonium sulfate and Potassium dihydrogen sulfate were auto-
claved in the reactor at a volume of 225 mL. Citric acid monohydrate was
dissolved in 5 mL of water, then sterile filtered through a 0.2 um syringe
filter. All other components and stock solutions were separately sterile
filtered or autoclaved. The remaining medium components were mixed
in a falcon tube in a sterile laminar air-flow bench and then
aseptically added to the MSTR with a syringe.

| Component | Target amount | Units |
|---|---|---|
| Ammonium sulfate ((NH₄)2SO₄) | 7.6 | g/L |
| Potassium dihydrogen sulfate | 15.7 | g/L |
| (KH₂PO₄) | | |
| Citric acid monohydrate (C₆H₈O_{7 ·} | 1.86 | g/L |
| H₂O) | | |
| Thiamine HCI stock | 10 | mL/L |
| (HC₁₂H₁₇ON₄SCl₂) | | |
| Trace metal stock 002 | 3.5 | mL/L |
| Trace metal stock 004 | 4 | mL/L |
| Magnesium sulfate (2M) | 1 | mL/L |
| (MgSO₄) | | |
| Glucose stock 20% (w/V) | 50 | mL/L |
| Water | Fill to desired volume | mL/L |

TABLE 3
Composition of the M9 salts stock solution used for pre-culture medium.
All components were dissolved in water and sterilized by autoclaving the
solution.

| Component | Target amount | Units |
|---|---|---|
| Disodium hydrogen phosphate | 68 | g/L |
| (Na₂HPO₄) | | |
| Potassium dihydrogen phosphate | 30 | g/L |
| (KH₂PO₄) | | |
| Sodium chloride (NaCl) | 5 | g/L |
| Ammonium chloride (NH₄Cl) | 10 | g/L |
| Water | Fill to desired volume | mL/L |

TABLE 4
Composition of the M9 Vitamin solution used for pre-culture medium.
All components were dissolved in water and sterilized by filtration.

| Component | Target amount | Units |
|---|---|---|
| Pyridoxine HCl (C₈H₁₂ClNO₃;) | 10 | mg/L |
| Thiamine HCl (C₁₂H₁₈Cl₂N₄OS) | 5 | mg/L |
| Riboflavin (Cₗ₇H₂₀N₄P₆) | 5 | mg/L |
| Nicotinic acid (C₆H₅NO₂) | 5 | mg/L |
| Ca-D-pantothenate (Cₗ₈H₃₂CaN₂P₁₀) | 5 | mg/L |
| p-aminobenzoic acid (CH₇NO₂) | 5 | mg/L |
| Thiotic acid (C₈H₁₄P₂S₂) | 5 | mg/L |
| Biotin (C₁₀H₁₆N₂O₃S) | 2 | mg/L |
| Folic acid (C₁₉H₁₉N₇O₆) | 2 | mg/L |
| Vitamin B12 (C₆₃H₈₈CoN₁₄O₁₄P) | 0.1 | mg/L |
| Water | Fill to desired volume | mL/L |

TABLE 5
Composition of the trace metal stock 002 used in the batch medium. All
components were dissolved in water and sterilized by autoclavation or
filtration.

| Component | Target amount | Units |
|---|---|---|
| Copper sulfate pentahydrate | 0.48 | g/L |
| (CuSO₄ · 5H₂O) | | |
| Cobalt chloride hexahydrate | 0.54 | g/L |
| (CoCl₂ · 6H₂O) | | |
| Zinc sulfate heptahydrate | 0.54 | g/L |
| (ZnSO₄ · 7H₂O) | | |
| Calcium chloride dihydrate | 2 | g/L |
| (CaCl₂ · 2H₂O) | | |
| Ferric chloride hexahydrate | 41.8 | g/L |
| (FeCl₃ · 6H₂O) | | |
| Manganese sulfate monohydrate | 0.3 | g/L |
| (MnSO₄ · H₂O) | | |
| EDTA disodium salt dihydrate | 33.4 | g/L |
| (C₁₀H₁₈N₂Na₂O₁₀) | | |
| Sodium molybdate dihydrate | 0.5 | g/L |
| (Na2MoO4 · 2H₂O) | | |
| Water | Fill to desired volume | mL/L |

TABLE 6
Composition of the trace metal stock 004 used in the batch medium. All
components were dissolved in water and sterilized by filtration.

| Component | Target amount | Units |
|---|---|---|
| Zinc sulfate heptahydrate | 5.4 | g/L |
| (ZnSO₄ · 7H₂O) | | |
| Water | Fill to desired volume | mL/L |

### Microorganism and Cultivation Conditions

The strain used in this study is a derivative of the Escherichia coli K12 wildtype BW25113 (Baba et al., 2006). Cells from glycerol −80° C. stocks prepared using the preculture medium (Table 1) were inoculated into a shake flask with 25 mL (250 mL shake flask) of the preculture medium (Table 1) and growth overnight at 30° C. and 200 rpm. The overnight grown culture was inoculated into the MSTR running with the batch medium (Table 2) at a starting OD₆₀₀ of 0.1 and grown until the cells reach a stationary phase Samples were taken periodically to monitor the growth of E. coli cells using the spectrophotometer by appropriate dilution using 0.9% NaCl solution. The growth profile of the cells was also monitored by the measurement of dissolved O₂ (using the Applikon Applisens DO₂ sensor), off-gas O₂ and CO₂ concentration using the off-gas mass spectrometer (PrimaBT, Thermofischer scientific). The pH of the medium was monitored using the Applikon Applisens pH sensor and controlled at a setpoint by base addition. The cultivation temperature was monitored using an externally mounted temperature sensor (TTS 500FA probe, Sensonic ApS, Denmark).

The setpoints for cultivation (Table 7) were chosen to accommodate an optimal growth condition for the E. coli strain. The ammonium hydroxide used for pH correction can also be used as nitrogen source by this strain. This would ensure that the growth could run unhindered until its completion. The reactor was operated through a cascade that will gradually increase the agitation to keep up with the O₂ requirement of the cells. Once the Dissolved O₂ (DO) decreases to 40%, the stirring will be increased by 50 rpm. This will happen every time when the DO decreases to 40% until the stirring is unable to increase more, at which point the aeration will increase instead, by 0.05 slpm every time when the DO decrease.

TABLE 7
Fermentation conditions for the MSTR to ensure optimal growth

| Setting | Setpoint |
|---|---|
| Reactor volume | 250 mL |
| Temperature | 30° C. |
| Aeration | 2 VVM airflow |
| Agitation | 500 RPM |
| DO% setpoint | 40% |
| pH | 6.5 |
| Base used | 15% NH₄OH |

### Results

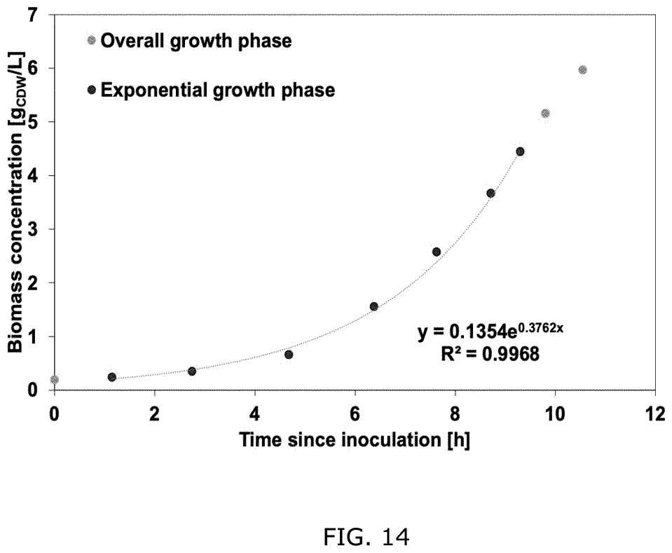

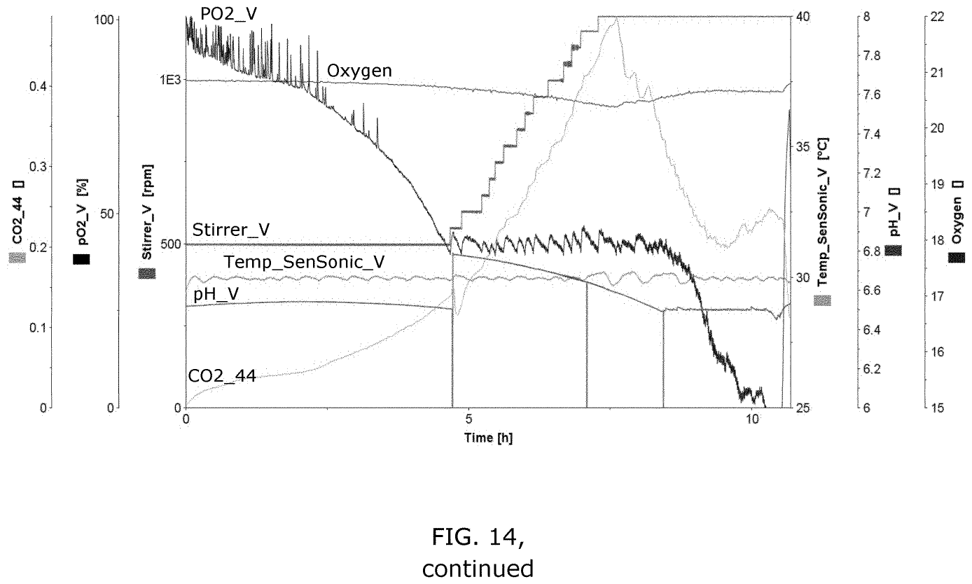

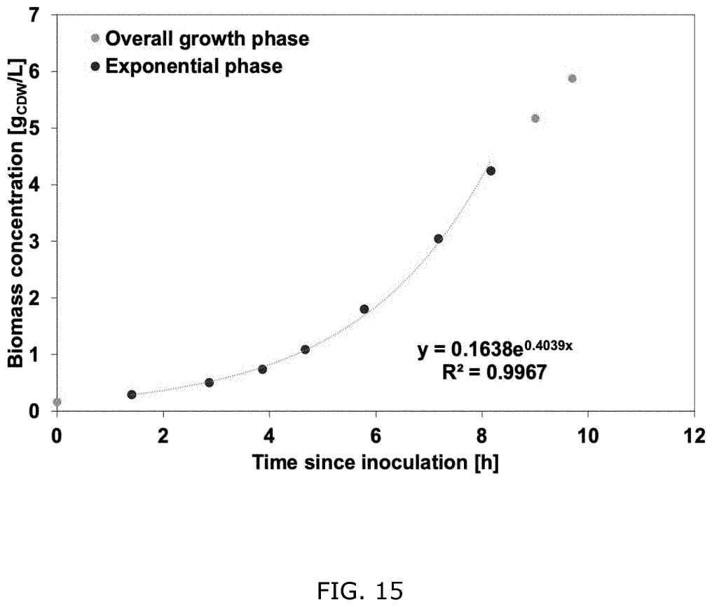

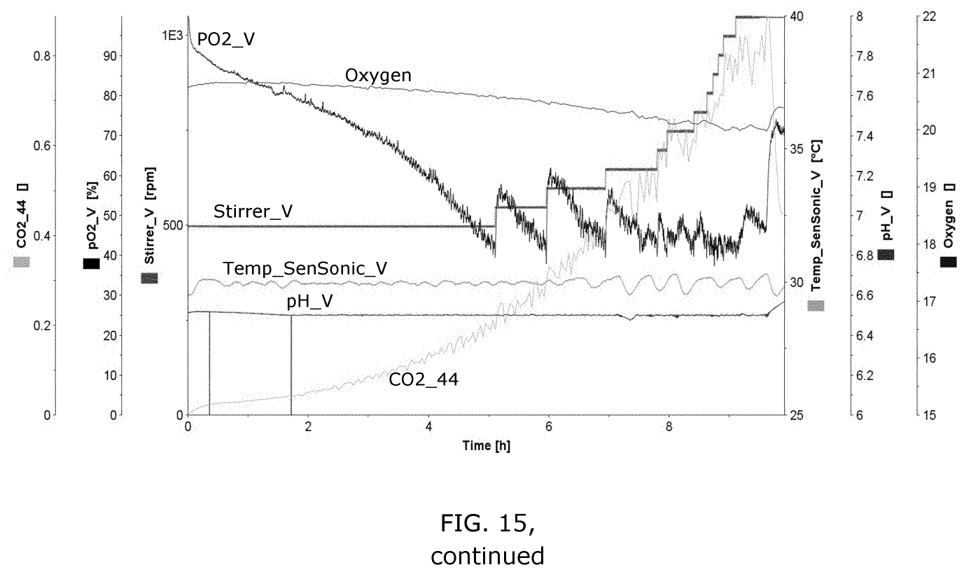

To test whether the MSTR is suitable for microbial cultivation, a controlled cultivation was performed with the bacterium E. coli in both the reference and scale-down configuration. The bacterium was able to grow well in MSTR to a biomass concentration of about 6 g_{CDW} L⁻¹ with a growth rate of 0.37 h⁻¹ (FIG. 14) and 0.40 h⁻¹ (FIG. 15). With a feedback control loop implemented in the BioCompact control unit, the parameters such as temperature, dissolved oxygen (DO) level and the pH was maintained at a constant value of 30° C., 40% (by increase of stirrer speed) and 6.5 (by the addition of base) throughout the cultivation time.

Suitability of the MSTR for scale-down operation

### Materials and Methods

To determine the liquid mixing time in MSTR, an experiment was set up where a buffered medium consisting of a mix of 2 M Succinic acid and 2 M Malonic acid¹ would be drip-fed 5 M Potassium hydroxide, and 5 M Sulfuric acid. The deviation in pH would be measured over a period of 5 minutes from each drop to determine the settling time, where 95% of the mixing had occurred. This was performed 3 times at each rpm setting, with and without aeration in the reactor and with different type of discs to observe the effect on liquid mixing.

$$C ( t ) = \frac{p H ( t ) - p {H}_{0}}{p {H}_{∞} - p {H}_{0}}$$

C(t) indicates the normalized pH at time t, where pH₀ is the initial pH before addition of base or acid. pH_{∞} is the steady pH where the mixing has settled and now reads a constant pH. This formula, combined with the formula for normalized time will be used to determine the time at which mixing has completed².

$$τ = \frac{t}{{t}_{95 \% mix}}$$

### Results

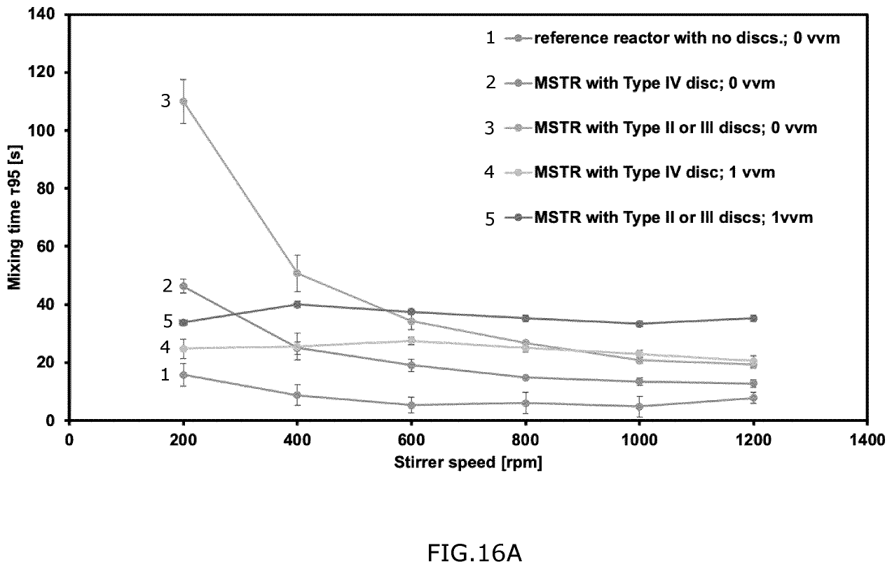

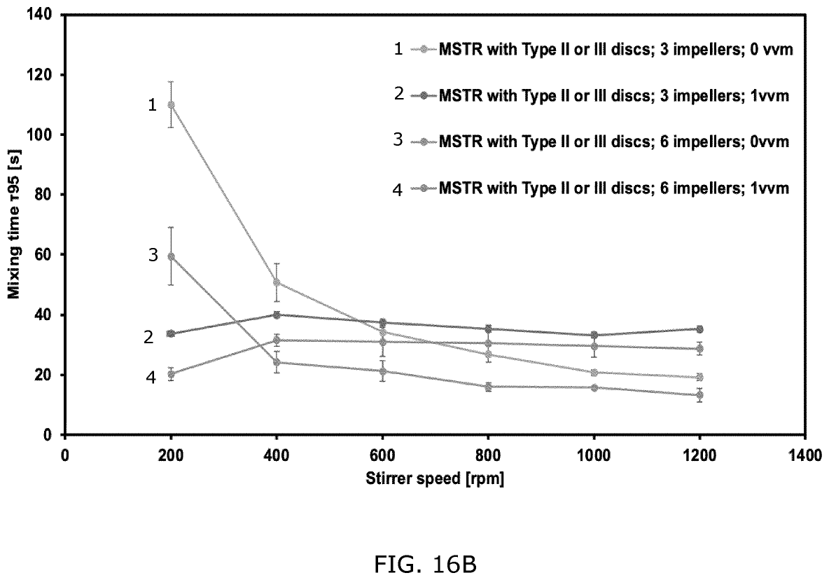

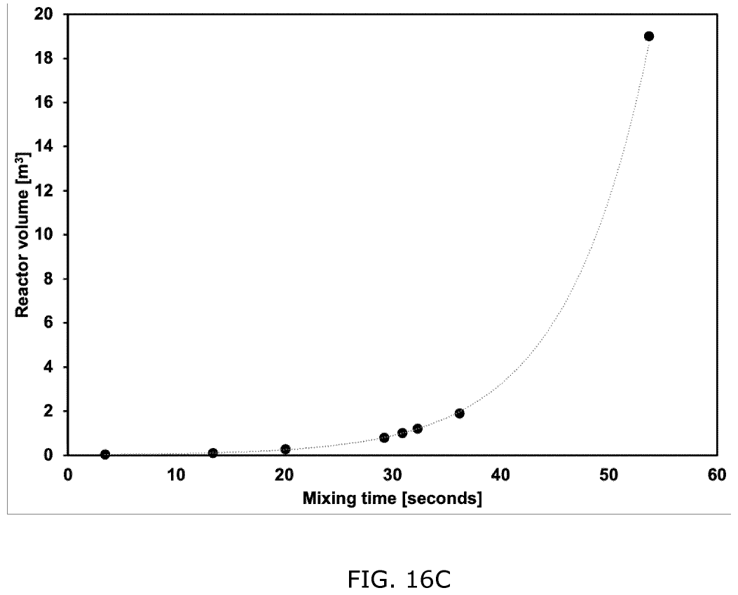

The operational flexibility of the MSTR to mimic mixing time of large-scale reactors has been improved with the current MSTR design, where a wide variety of liquid mixing time i.e., from 5 to 60 seconds (with aeration) and to 110 seconds (without aeration) can be obtained depending upon the process needs in a small-scale cultivation volume of 250-350 mL (FIG. 16).

### REFERENCES

Baba, T. et al. Construction of Escherichia coli K-12 in-frame, single-gene knockout mutants: the Keio collection. Mol. Syst. Biol. 2, 2006.0008 (2006).
Dragosits, M., Mattanovich, D. Adaptive laboratory evolution—principles and applications for biotechnology. Microb Cell Fact 12, 64 (2013). https://doi.org/10.1186/1475-2859-12-64
Gaugler et al. 2022. Scaling-down biopharmaceutical production processes via a single multi-compartment bioreactor (SMCB). Engineering in Life Sciences. https://doi.org/10.1002/elsc.202100161
Haringa, C., 2019. Through the Organism's eyes. The interaction between hydrodynamics and metabolic dynamics in industrial-scale fermentation processes. TU Delft Univ. doi:10.4233/uuid
Junker, B. H., 2004. Scale-up methodologies for Escherichia coli and yeast fermentation processes. J Biosci Bioeng 97, 347-364.
Lee D H, Feist A M, Barrett C L, Palsson B0 (2011) Cumulative Number of Cell Divisions as a Meaningful Timescale for Adaptive Laboratory Evolution of Escherichia coli. PLoS ONE 6(10): e26172. https://doi.org/10.1371/journal.pone.0026172
Neubauer, P., Junne, S., 2010. Scale-down simulators for metabolic analysis of large-scale bioprocesses. Curr. Opin. Biotechnol. 21, 114-121. doi:10.1016/j.copbio.2010.02.001
Nørregaard, A., Bach, C., KrQhne, U., Borgbjerg, U. & Gernaey, K. V. Hypothesis-driven compartment model for stirred bioreactors utilizing computational fluid dynamics and multiple pH sensors. Chem. Eng. J. 356, 161-169 (2019).
Oosterhuis, N. M. G., 1984. Scale-up of bioreactors: a scale-down approach. TU Delft.
Poulsen, B. R. & Iversen, J. J. L. Mixing determinations in reactor vessels using linear buffers. Chem. Eng. Sci. 52, 979-984 (1997).
Schilling, B. M., Pfefferle, W., Bachmann, B., Leuchtenberger, W. & Deckwer, W. D. A special reactor design for investigations of mixing time effects in a scaled-down industrialL-lysine fed-batch fermentation process. Biotechnol. Bioeng. 64, 599-606 (1999).
WO/2012/097079. Tuohey et al. LINEARLY SCALABLE SINGLE USE BIOREACTOR SYSTEM

### Items

1. A scale-down reactor comprising:
   a. a reaction compartment comprising at least two reactor units, wherein said at least two reactor units are reversibly attached to one another via connection means,
      wherein the at least two reactor units comprise a top unit (1) and a bottom unit (2), and optionally one or more further inner reactor units (3);
      wherein said reactor units when attached to one another form a fluid-tight reaction compartment configured to receive a reaction liquid therein;
   b. one or more inlet apertures (4),
   c. one or more outlet apertures (4),
   d. optionally, a mixing shaft (5) configured to be placed vertically in the reaction compartment, said mixing shaft comprising one or more impellers (6), and
   e. at least one disk (7) configured to be inserted horizontally between two adjacent reactor units or horizontally within an inner reaction unit, said disk comprising at least one aperture (16) allowing the fluid contained in the adjacent reactor units to pass through it, optionally said disk comprising at least one shaft aperture (8) allowing the mixing shaft to pass through it.
2. A scale-down reactor comprising:
   a. a reaction compartment comprising at least two reactor units, wherein said at least two reactor units are reversibly attached to one another via connection means,
      wherein the at least two reactor units comprise a top unit (1) and a bottom unit (2), and optionally one or more further inner reactor units (3);
      wherein said reactor units when attached to one another form a fluid-tight reaction compartment for receiving a reaction liquid therein;
   b. one or more inlet apertures (4),
   c. one or more outlet apertures (4),
   d. optionally, a mixing shaft (5) placed vertically in the reaction compartment, said mixing shaft comprising one or more impellers (6), and
   e. at least one disk (7) inserted horizontally between two adjacent reactor units or horizontally within an inner reaction unit, said disk comprising at least one aperture (16) allowing the fluid contained in the adjacent reactor units to pass through it, optionally said disk comprising at least one shaft aperture (8) allowing the mixing shaft to pass through it.
3. The scale-down reactor according to any one of the preceding items, wherein the space between two adjacent disks or between one disk and the top or the bottom of the reactor defines a sub-volume from the total volume of the reactor.
4. The scale-down reactor according to any one of the preceding items, wherein said reactor comprises between 2 and 10 reactor units, such as between 3 and 8 reactor units, such as between 4 and 7 rector units, such as between 5 and 6 reactor units.
5. The scale-down reactor according to any one of the preceding items, wherein said reactor comprises at least 3 reactor units, such as at least 4 reactor units, such as at least 5 reactor units, such as at least 6 reactor units, such as at least 7 reactor units, such as at least 8 reactor units, such as at least 9 reactor units, such as at least 10 reactor units.
6. The scale-down reactor according to any of the preceding items, wherein the total volume of the reactor is reversibly modified by varying the number of inner reactor units.
7. The scale-down reactor according to any one of the preceding items, wherein the reactor is a stirred tank reactor or a bubble column reactor.
8. The scale-down reactor according to any one of the preceding items, wherein the total volume of the reactor is between 60 mL and 35 L, such as between 100 mL and 30 L, such as between 500 mL and 15 L, such as between 1 L and 10 L, such as between 60 mL and 700 mL.
9. The scale-down reactor according to any one of the preceding items, wherein the working volume of the reactor is at most 90% of the total volume of the reactor, wherein said working volume is defined as the amount of reaction liquid contained in said reactor.
10. The scale-down reactor according to any one of the preceding items, wherein the working volume of the reactor is between 50 mL and 30 L, such as between 100 mL and 30 L, such as between 500 mL and 15 L, such as between 1 L and 10 L, such as between 50 mL and 600 mL.
11. The scale-down reactor according to any one of items 3 to 10, wherein the sub-volume is between 50 and 600 mL, such as between 50 and 500 mL, such as between 50 mL and 400 mL, such as between 50 mL and 300 mL, such as between 50 mL and 200 mL, such as between 50 mL and 100 mL, such as between 50 mL and 60 mL, such as between 100 and 600 mL, such as between 200 and 500 mL.
12. The scale-down reactor according to any one of the preceding items, wherein the height of said reactor is reversibly modified by varying the number of inner reactor units.
13. The scale-down reactor according to any one of the preceding items, wherein the height of the reactor is between 130 mm and 800 mm, such as between 130 mm and 700 mm, such as between 130 mm and 600 mm, such as between 130 mm and 500 mm, such as between 130 mm and 400 mm, such as between 130 mm and 300 mm, such as between 350 mm and 750 mm, such as between 400 mm and 800 mm.
14. The scale-down reactor according to any one of the preceding items, wherein the inner diameter of the reactor is between 50 mm and 150 mm, such as between 60 mm and 140 mm, such as between 60 mm and 130 mm.
15. The scale-down reactor according to any one of the preceding items, wherein the aspect ratio of the reactor is between 1.6 and 4.2, such as 1.6, 2.0, 2.5, 3.0, 3.5, 4.0 or 4.2, wherein said aspect ratio is defined as the reactor inner height divided with the reactor inner diameter.
16. The scale-down reactor according to any of the preceding items, wherein the connection means are reversibly and mechanically engaging elements.
17. The scale-down reactor according to item 16, wherein the engaging elements are latches, fastening objects, preferably screw-like mechanisms or more preferably clicking screw-like mechanisms.
18. The scale-down reactor according to item 17, wherein the engaging elements are plugs.
19. The scale-down reactor according to any of the preceding items, wherein one or more reactor units comprises an observation aperture (9), preferably wherein the top unit comprises an observation aperture.
20. The scale-down reactor according to any of the preceding items, wherein the one or more inlet apertures allow for the injection and/or sampling of liquids, solids or gases, preferably in a fluid-tight manner.
21. The scale-down reactor according to any of the preceding items, wherein the one or more outlet apertures allow for the injection and/or sampling of liquids, solids or gases, preferably in a fluid-tight manner.
22. The scale-down reactor according to any of the preceding items, wherein at least one reactor unit comprises one or more inlet and/or outlet apertures, such as wherein at least two reactor units comprises one or more inlet and/or outlet apertures, such as wherein at least three reactor units comprises one or more inlet and/or outlet apertures, such as wherein at least four reactor units comprises one or more inlet and/or outlet apertures, such as wherein at least five reactor units comprises one or more inlet and/or outlet apertures, such as wherein at least six reactor units comprises one or more inlet and/or outlet apertures, such as wherein at least seven reactor units comprises one or more inlet and/or outlet apertures, such as wherein all reactor units comprises one or more inlet and/or outlet apertures, allowing for the injection and/or sampling of liquids, solids or gases, preferably in a fluid-tight manner.
23. The scale-down reactor according to any one of the preceding items, wherein the top unit comprises at least four inlet and/or outlet apertures.
24. The scale-down reactor according to any one of the preceding items, wherein the bottom unit comprises inlet apertures for injection of gases, such as inlet apertures for aerating the reaction liquid.
25. The scale-down reactor according to any one of the preceding items, wherein said reactor comprises one or more inner reactor unit(s) and wherein at least one of said inner reactor units comprises one or more inlet and/or outlet apertures, such as wherein all of said one or more inner reactor unit(s) comprises one or more inlet and/or outlet apertures.
26. The scale-down reactor according to any of the preceding items, wherein the reactor comprises a temperature control jacket (10) comprising one or more jacket units (11, 12, 13), said control jacket being configured for modifying the temperature of the reaction fluid contained in the reactor.
27. The scale-down reactor according to item 26, wherein the temperature control jacket comprises a top unit (11) and a bottom unit (12) and optionally one or more middle jacket units (13), wherein the total jacket height is reversibly modified by varying the number of jacket units, and further wherein said jacket encloses at least the bottom unit and the optional one or more further inner reactor units.
28. The scale-down reactor according to item 27, wherein the jacket units are reversibly attached to one another via connection means.
29. The scale-down reactor according to any of items 26 to 28, wherein the temperature control jacket comprises one inlet aperture (14) and one outlet aperture (15) to allow for water to flow through the jacket.
30. The scale-down reactor according to any of items 3 to 29, comprising a mixing shaft, wherein the mixing shaft comprises an impeller in at least one sub-volume, such as in at least two sub-volumes, such as in at least three sub-volumes, such as in at least four sub-volumes, such as in at least five sub-volumes, such as in at least six sub-volumes, such as in at least seven sub-volumes, such as in each inner sub-volume.
31. The scale-down reactor according to item 30, wherein the impeller is selected from the group consisting of an axial flow impeller, a tangential flow impeller, a radial flow impeller, a high shear impeller and a specialty impeller or combinations thereof, such as a Rushton turbine, cross beam, frame impeller, anchor impeller, flat-blade impeller, pointe blade impeller, propeller, pitched blade turbine, hydrofoils, hollow-blade turbine, helical ribbon, cowls, disk, bar, pointed blade impeller, retreat curve impeller, sweptback impeller, spring impeller, or a glass-lined turbine.
32. The scale-down reactor according to any of the preceding items, wherein the at least one disk is reversibly inserted transversally to the main axis of the reactor unit.
33. The scale-down reactor according to any of the preceding items, wherein the at least one disk separates the sub-volumes created by adjacent reactor units.
34. The scale-down reactor according to anyone of the preceding items, wherein the disk comprises at least 1 aperture, such as at least 2 apertures, such as at least 3 apertures, such as at least 4 apertures, such as at least 5 apertures, such as at least 6, such as at least 7 apertures, such as at least 8 apertures, such as at least 9 apertures, such as at least 10 apertures, such as at least 11, such as at least 12 apertures, such as at least 13 apertures, such as at least 14 apertures, such as at least 15 apertures, such as at least 16, such as at least 17 apertures, such as at least 18 apertures, such as at least 19 apertures, such as at least 20 apertures, such as at least 25, such as at least 30 apertures, such as at least 35 apertures, such as at least 40 apertures, such as at least 45 apertures, such as at least 50 apertures, such as at least 60 apertures, such as at least 70 apertures, such as at least 80 apertures, such as at least 90 apertures, such as at least 100 apertures, such as at least 200 apertures, such as at least 300 apertures, such as at least 400 apertures, such as at least 500 apertures, such as at least 600 apertures, such as at least 700 apertures, such as at least 800 apertures, such as at least 900 apertures, such as at least 1000 apertures, such as at least 1100 apertures, such as at least 1200 apertures, such as at least 1300 apertures, such as at least 1400 apertures, such as at least 1500 apertures.
35. The scale-down reactor according to anyone of the preceding items, wherein the disk comprises between 1 and 1500 apertures, such as between 1 and 1000 apertures, such as between 5 and 500 apertures, such as between 10 and 100 apertures, such as between 500 and 1000 apertures, such as between 100 and 1000 apertures, such as between 5 and 50 apertures, such as between 10 and 50 apertures, such as between 20 and 200 apertures, such as between 200 and 800 apertures, such as between 2 and 1000 apertures, such as between 3 and 300 apertures.
36. The scale-down reactor according to anyone of the preceding items, wherein the apertures are of different size and shape, preferably wherein the apertures are circular.
37. The scale-down reactor according to anyone of the preceding items, wherein the apertures are circular and further wherein said apertures are between 0.5 mm and 100 mm in diameter, such as between 50 mm and 100 mm in diameter, such as between 0.5 mm and 50 mm in diameter, such as between 0.5 mm and 40 mm in diameter, such as between 0.5 mm and 30 mm in diameter, such as between 0.5 mm and 20 mm in diameter, such as between 0.5 mm and 10 mm in diameter, such as between 0.5 mm and 5 mm in diameter, such as between 0.5 mm and 4 mm in diameter, such as between 0.5 mm and 3 mm in diameter, such as between 0.5 mm and 2 mm in diameter, such as between 0.5 mm and 1 mm in diameter, such as between 1 mm and 2 mm in diameter, such as between 2 mm and 100 mm in diameter, such as between 2 mm and 50 mm in diameter, such as between 2 mm and 40 mm in diameter, such as between 2 mm and 30 mm in diameter, such as between 2 mm and 20 mm in diameter, such as between 2 mm and 10 mm in diameter.
38. The scale-down reactor according to anyone of the preceding items, wherein the size, shape and number of apertures is selected to provide a specific exchange area between the sub-volumes of adjacent reactor units.
39. The scale-down reactor according to any one of the preceding items, wherein the size, shape, number and location of the apertures, and/or the impeller, is selected to provide a specific flow profile between the sub-volumes of adjacent reactor units.
40. The scale-down reactor according to any one of items 38 to 39, wherein the specific exchange area and/or the specific flow profile between the sub-volumes of adjacent reactor units are designed to provide a specific mixing time in each sub-volume of each adjacent reactor unit, preferably wherein said mixing time is different in each sub-volume of adjacent reactor units.
41. The scale-down reactor according to item 40, wherein the mixing time of a sub-volume of a reactor unit is defined as the time required for achieving a pre-determined degree of homogeneity in said sub-volume of said reactor unit.
42. The scale-down reactor according to any one of items 40 to 41, wherein the mixing time of a sub-volume of a reactor unit further is dependent on the volumetric power input.
43. The scale-down reactor according to any of the preceding items, wherein the reactor units are manufactured from stainless steel or a polymer, preferably wherein the stainless steel is of type 306 or 306 L and the polymer is polycarbonate.
44. The scale-down reactor according to any of the preceding items, wherein the top unit and/or the bottom unit are manufactured from stainless steel, and wherein the one or more further inner reactor units are manufactured from a polymer, preferably wherein the stainless steel is of type 306 or 306 L and the polymer is polycarbonate.
45. The scale-down reactor according to any one of items 43 to 44, wherein the polycarbonate is Sustanat® PC.
46. A method of simulating, modelling, monitoring or modifying the conditions of a reactor comprising a liquid composition in a bioreactor or live fermentation process, comprising the steps of:
   a. providing the scale-down reactor according to any one of the preceding items;
   b. defining the total reactor volume by reversibly adding a number of inner reactor units (3);
   c. defining the number, position and design of the at least one disk (7); and
   d. optionally, defining the design of the mixing shaft (5) and one or more impeller (6);
   wherein the conditions to be simulated, modelled, monitored or modified are selected from the group consisting of: reaction volume, temperature, pH, gas concentration, metabolite concentration, cellular mass, cellular density, enzymatic kinetics, absorbance, and mixing time, and wherein the reactor is at least 300 L.
47. The method according to item 46, wherein one or more of the conditions is simulated, modelled, monitored or modified in each sub-volume of the scale-down reactor.
48. The method according to any one of items 46 to 47, wherein one or more operating conditions in the scale-down reactor are equal or similar to said corresponding operating conditions in the reactor, wherein said operating conditions are selected from:
   a. Power input value (P/V);
   b. aspect ratio;
   c. impeller tip speed;
   d. mixing time;
   e. volumetric mass transfer coefficient; and/or
   f. oxygen transfer rate.
49. The method according any one of items 46 to 47, wherein the Power Input Value (P/V) of the scale-down reactor is equal or similar to the P/V of the reactor, wherein P is the Power Input and V is the Volume in the reactor, and wherein P may be calculated according to the formula:

$$P = Po ⋆ ρ ⋆ N ⋆ {D}^{3}$$

wherein V is the volume of the reaction liquid, Po is the Power number for the impeller, ρ is the fluid density, N is the rotational speed of the impeller and D is the impeller diameter.
50. The method according any one of items 46 to 49, wherein the scale-down reactor is operated in the same mode as the reactor, such as in batch, fed-batch or continuous mode, and further wherein the scale-down reactor is of the same type as the reactor, wherein the type is selected from a stirred tank reactor and a bubble column reactor.
51. The method according any one of items 46 to 50, wherein the volumetric mass transfer coefficient (k_{L}a) and/or the oxygen transfer rate (OTR) of the scale-down reactor is equal or similar to the k_{L}a and/or the OTR of the reactor, wherein said coefficient and rate may be calculated according to the formula:

$${k}_{L} a = OTR / ( {C}_{L}^{⋆} - {C}_{L} )$$

wherein C*_{L} is the dissolved oxygen concentration at air saturation and C_{L} is the dissolved oxygen concentration in the liquid phase.
52. The method according any one of items 46 to 51, wherein the mixing time of the scale-down reactor is equal or similar to the mixing time of the reactor, wherein said mixing time is defined as the time required for achieving a pre-determined degree of homogeneity in the reactor.
53. The method according to any one of items 46 to 52, wherein the method comprises cultivation of cells, such as eukaryotic or prokaryotic cells, such as Chinese Hamster Ovary (CHO) cells, yeast, fungi or bacteria.
54. Use of a scale-down reactor according to any one of items 1 to 43 for simulating, modelling, monitoring or modifying the conditions of a reactor, wherein the conditions to be simulated, modelled, monitored or modified are selected from the group consisting of: reaction volume, temperature, pH, gas concentration, metabolite concentration, cellular mass, cellular density, enzymatic kinetics, absorbance, and mixing time,
   wherein the reactor is at least 300 L.
55. The use according to item 54, wherein the reactor is a bioreactor or a live fermentation process.
56. The use according to item 55, wherein the bioreactor or live fermentation process comprises the cultivation of cells, such as eukaryotic or prokaryotic cells, such as Chinese Hamster Ovary (CHO) cells, yeast, fungi or bacteria.
57. The use according to any one of items 54 to 56, wherein one or more of the conditions is simulated, modelled, monitored or modified in each sub-volume of the scale-down reactor.
58. The use according to item any one of items 54 to 57, wherein one or more operating conditions in the scale-down reactor are equal or similar to said corresponding operating conditions in the reactor, wherein said operating conditions are selected from:
   a. Power input value (P/V);
   b. aspect ratio;
   c. impeller tip speed;
   d. mixing time;
   e. volumetric mass transfer coefficient; and/or
   f. oxygen transfer rate.
59. The use according to item any one of items 54 to 58, wherein the Power Input Value (P/V) of the scale-down reactor is equal or similar to the P/V of the reactor, wherein P is the Power Input and V is the Volume in the reactor, and wherein P may be calculated according to the formula:

$$P = Po ⋆ ρ ⋆ N ⋆ {D}^{3}$$

wherein V is the volume of the reaction liquid, Po is the Power number for the impeller, ρ is the fluid density, N is the rotational speed of the impeller and D is the impeller diameter.
60. The use according to item any one of items 54 to 59, wherein the aspect ratio of the scale-down reactor is equal or similar to the aspect ratio of the reactor.
61. The use according to item any one of items 54 to 60, wherein the volumetric mass transfer coefficient (k_{L}a) and/or the oxygen transfer rate (OTR) of the scale-down reactor is equal or similar to the k_{L}a and/or the OTR of the reactor, wherein said coefficient and rate may be calculated according to the formula:

$${k}_{L} a = OTR / ( {C}_{L}^{⋆} - {C}_{L} )$$

wherein C*_{L} is the dissolved oxygen concentration at air saturation and C_{L} is the dissolved oxygen concentration in the liquid phase.
62. The use according to item any one of items 54 to 61, wherein the mixing time of the scale-down reactor is equal or similar to the mixing time of the scale-down reactor, wherein said mixing time is defined as the time required for achieving a pre-determined degree of homogeneity in the reactor.
63. The use according to item any one of items 54 to 62, wherein the scale-down reactor is operated in the same mode as the reactor, such as in batch, fed-batch or continuous mode, and further wherein the scale-down reactor is of the same type as the reactor, wherein the type is selected from a stirred tank reactor and a bubble column reactor.
64. Use of the stirred tank reactor according to any one of items 1 to 43 for liquid-liquid extraction.
65. A method of adapting a microorganism to the conditions of a reactor, said method comprising:
   a. providing the scale-down reactor according to any one of items 1 to 43;
   b. defining the total reactor volume by reversibly adding a number of inner reactor units (3);
   c. defining the number, position and design of the at least one disk (7);
   d. optionally, defining the design of the mixing shaft (5) and one or more impellers (6); and
   e. growing the microorganism in the scale-down reactor for an extended amount of time in order to allow the microorganism to adapt to the conditions of the reactor;
wherein the reactor is at least 300 L, thereby obtaining an adapted microorganism.
66. The method according to item 65, wherein adapting the microorganism comprises the microorganism acquiring one or more adaptive mutations.
67. The method according to any one of items 65 to 66, wherein the conditions are selected from the group consisting of: fluctuating temperature, fluctuating pH, fluctuating metabolite concentration, fluctuating gas concentration, and fluctuating oxygen concentration.
68. The method according to any one of items 65 to 67, wherein the adapted microorganism has improved characteristics, such as wherein the adapted microorganism has a higher growth rate when compared to a similar non-adapted microorganism when grown under the same conditions.
69. The method according to any one of items 65 to 68, wherein the microorganism is grown in the reactor for at least 101 cumulative number of cell divisions.
70. A system comprising the scale-down reactor according to any one of items 1 to 43 and a reaction liquid.
71. A method of manufacturing a scale-down reactor, said method comprising:
   a. providing
      i. a reaction compartment comprising at least two reactor units, wherein said at least two reactor units are reversibly attached to one another via connection means,
      wherein the at least two reactor units comprise a top unit (1) and a bottom unit (2), and optionally comprise one or more further inner reactor units (3); wherein said reactor units when attached to one another form a fluid-tight reaction compartment configured to receive a reaction liquid therein; wherein one or more of said units comprise one or more inlet fluid apertures (4) and one or more outlet fluid apertures (4),
      ii. optionally, a mixing shaft (5) configured to be placed vertically in the reaction compartment, said mixing shaft comprising one or more impellers (6), and
      iii. at least one disk (7) configured to be inserted horizontally between two adjacent reactor units or horizontally within an inner reaction unit, said disk comprising at least one aperture (16) allowing the fluid contained in the adjacent reactor units to pass through it, optionally said disk comprising at least one shaft aperture (8) allowing the mixing shaft to pass through it;
   b. assembling said reactions units, disk and optionally said mixing shaft.
72. A method of manufacturing a scale-down reactor, said method comprising:
   a. providing
      i. a reaction compartment comprising at least two reactor units, wherein said at least two reactor units are reversibly attached to one another via connection means,
      wherein the at least two reactor units comprise a top unit (1) and a bottom unit (2), and optionally comprise one or more further inner reactor units (3); wherein said reactor units when attached to one another form a fluid-tight reaction compartment for receiving a reaction liquid therein; wherein one or more of said units comprise one or more inlet fluid apertures (4) and one or more outlet fluid apertures (4),
      ii. optionally, a mixing shaft (5) placed vertically in the reaction compartment, said mixing shaft comprising one or more impellers (6), and
      iii. at least one disk (7) inserted horizontally between two adjacent reactor units or horizontally within an inner reaction unit, said disk comprising at least one aperture (16) allowing the fluid contained in the adjacent reactor units to pass through it, optionally said disk comprising at least one shaft aperture (8) allowing the mixing shaft to pass through it;
   b. assembling said reactions units, disk and optionally said mixing shaft.
73. A method of controlling a scale-down reactor, said method comprising:
   a. providing
      i. a reaction compartment comprising at least two reactor units, wherein said at least two reactor units are reversibly attached to one another via connection means,
         wherein the at least two reactor units comprise a top unit (1) and a bottom unit (2), and optionally comprise one or more further inner reactor units (3); wherein said reactor units when attached to one another form a fluid-tight reaction compartment for receiving or configured for receiving a reaction liquid therein; wherein one or more of said units comprise one or more inlet fluid apertures (4) and one or more outlet fluid apertures (4),
      ii. optionally, a mixing shaft (5) placed vertically or configured to be placed vertically in the reaction compartment, said mixing shaft comprising one or more impellers (6), and
      iii. at least one disk (7) inserted horizontally or configured to be inserted horizontally between two adjacent reactor units or horizontally within an inner reaction unit, said disk comprising at least one aperture (16) allowing the fluid contained in the adjacent reactor units to pass through it, optionally said disk comprising at least one shaft aperture (8) allowing the mixing shaft to pass through it;
      iv. at least one probe located in at least one of the reactor units, configured to measure at least one property of the reactor, such as pH, dissolved oxygen, temperature, real-time flow cytometry and/or concentration of one or more products present inside the reactor,
   b. assembling said reactions units, disk and optionally said mixing shaft,
   c. providing a computing unit containing a digital representation of the scale-down reactor,
   d. receiving data from the at least one probe, and
e. varying operating conditions of the scale-down reactor until a simulated optimal state provided by the digital representation of the scale-down reactor is achieved.
74. The method according to item 73, wherein the digital representation of the scale-down reactor is indistinguishable from the scale-down reactor.
75. The method according to any one of items 73 to 74, wherein the digital representation of the scale-down reactor is a modelling system, such as a digital twin model, a machine learning model or a neural network model.

1. A scale-down reactor comprising:
a. a reaction compartment comprising at least two reactor units, wherein said at least two reactor units are reversibly attached to one another via connection means,
wherein the at least two reactor units comprise a top unit (1) and a bottom unit (2), and optionally one or more further inner reactor units (3);
wherein said reactor units when attached to one another form a fluid-tight reaction compartment for receiving a reaction liquid therein;
b. one or more inlet apertures (4),
c. one or more outlet apertures (4),
d. optionally, a mixing shaft (5) placed vertically in the reaction compartment, said mixing shaft comprising one or more impellers (6), and
at least one disk (7) inserted horizontally between two adjacent reactor units or horizontally within an inner reaction unit, said disk comprising at least one aperture (16) allowing the fluid contained in the adjacent reactor units to pass through it, optionally said disk comprising at least one shaft aperture (8) allowing the mixing shaft to pass through it.

2. The scale-down reactor according to claim 1, wherein the space between two adjacent disks or between one disk and the top or the bottom of the reactor defines a sub-volume from the total volume of the reactor.

3. The scale-down reactor according to any one of the preceding claims, wherein the total volume of the reactor is reversibly modified by varying the number of inner reactor units, optionally wherein said reactor comprises between 2 and 10 reactor units, such as between 3 and 8 reactor units, such as between 4 and 7 rector units, such as between 5 and 6 reactor units, further optionally wherein the reactor comprises at least 3 reactor units, such as at least 4 reactor units, such as at least 5 reactor units, such as at least 6 reactor units, such as at least 7 reactor units, such as at least 8 reactor units, such as at least 9 reactor units, such as at least 10 reactor units.

4. The scale-down reactor according to any one of the preceding claims, wherein the total volume of the reactor is reversibly modified by varying the number of inner reactor units, optionally wherein the aspect ratio of the reactor is between 1.6 and 4.2, such as 1.6, 2.0, 2.5, 3.0, 3.5, 4.0 or 4.2, wherein said aspect ratio is defined as the reactor inner height divided with the reactor inner diameter.

5. The scale-down reactor according to any of the preceding claims, wherein the connection means are reversibly and mechanically engaging elements, optionally wherein the engaging elements are latches, fastening objects, preferably screw-like mechanisms or more preferably clicking screw-like mechanisms, further optionally wherein the engaging elements are plugs.

6. The scale-down reactor according to any of the preceding claims, wherein the reactor comprises a temperature control jacket (10) comprising one or more jacket units (11, 12, 13), said control jacket being configured for modifying the temperature of the reaction fluid contained in the reactor,
optionally wherein the temperature control jacket comprises a top unit (11) and a bottom unit (12) and optionally one or more middle jacket units (13), wherein the total jacket height is reversibly modified by varying the number of jacket units, and further wherein said jacket encloses at least the bottom unit and the optional one or more further inner reactor units,
further optionally wherein the jacket units are reversibly attached to one another via connection means, and
even further optionally wherein the temperature control jacket comprises one inlet aperture (14) and one outlet aperture (15) to allow for water to flow through the jacket.

7. The scale-down reactor according to any of claims 2 to 6, comprising a mixing shaft, wherein the mixing shaft comprises an impeller in at least one sub-volume, such as in at least two sub-volumes, such as in at least three sub-volumes, such as in at least four sub-volumes, such as in at least five sub-volumes, such as in at least six sub-volumes, such as in at least seven sub-volumes, such as in each inner sub-volume,
optionally wherein the impeller is selected from the group consisting of an axial flow impeller, a tangential flow impeller, a radial flow impeller, a high shear impeller and a specialty impeller or combinations thereof, such as a Rushton turbine, cross beam, frame impeller, anchor impeller, flat-blade impeller, pointe blade impeller, propeller, pitched blade turbine, hydrofoils, hollow-blade turbine, helical ribbon, cowls, disk, bar, pointed blade impeller, retreat curve impeller, sweptback impeller, spring impeller, or a glass-lined turbine.

8. The scale-down reactor according to any of the preceding claims, wherein the at least one disk is reversibly inserted transversally to the main axis of the reactor unit,
optionally wherein the at least one disk separates the sub-volumes created by adjacent reactor units.

9. The scale-down reactor according to anyone of the preceding claims, wherein the disk comprises at least 1 aperture, such as at least 2 apertures, such as at least 3 apertures, such as at least 4 apertures, such as at least 5 apertures, such as at least 6, such as at least 7 apertures, such as at least 8 apertures, such as at least 9 apertures, such as at least 10 apertures, such as at least 11, such as at least 12 apertures, such as at least 13 apertures, such as at least 14 apertures, such as at least 15 apertures, such as at least 16, such as at least 17 apertures, such as at least 18 apertures, such as at least 19 apertures, such as at least 20 apertures, such as at least 25, such as at least 30 apertures, such as at least 35 apertures, such as at least 40 apertures, such as at least 45 apertures, such as at least 50 apertures, such as at least 60 apertures, such as at least 70 apertures, such as at least 80 apertures, such as at least 90 apertures, such as at least 100 apertures, such as at least 200 apertures, such as at least 300 apertures, such as at least 400 apertures, such as at least 500 apertures, such as at least 600 apertures, such as at least 700 apertures, such as at least 800 apertures, such as at least 900 apertures, such as at least 1000 apertures, such as at least 1100 apertures, such as at least 1200 apertures, such as at least 1300 apertures, such as at least 1400 apertures, such as at least 1500 apertures,
optionally wherein the apertures are circular and further wherein said apertures are between 0.5 mm and 100 mm in diameter, such as between 50 mm and 100 mm in diameter, such as between 0.5 mm and 50 mm in diameter, such as between 0.5 mm and 40 mm in diameter, such as between 0.5 mm and 30 mm in diameter, such as between 0.5 mm and 20 mm in diameter, such as between 0.5 mm and 10 mm in diameter, such as between 0.5 mm and 5 mm in diameter, such as between 0.5 mm and 4 mm in diameter, such as between 0.5 mm and 3 mm in diameter, such as between 0.5 mm and 2 mm in diameter, such as between 0.5 mm and 1 mm in diameter, such as between 1 mm and 2 mm in diameter, such as between 2 mm and 100 mm in diameter, such as between 2 mm and 50 mm in diameter, such as between 2 mm and 40 mm in diameter, such as between 2 mm and 30 mm in diameter, such as between 2 mm and 20 mm in diameter, such as between 2 mm and 10 mm in diameter, further optionally wherein the size, shape and number of apertures is selected to provide a specific exchange area between the sub-volumes of adjacent reactor units,
further optionally wherein the size, shape, number and location of the apertures, and/or the impeller, is selected to provide a specific flow profile between the sub-volumes of adjacent reactor units,
even further optionally wherein the specific exchange area and/or the specific flow profile between the sub-volumes of adjacent reactor units are designed to provide a specific mixing time in each sub-volume of each adjacent reactor unit, preferably wherein said mixing time is different in each sub-volume of adjacent reactor units and wherein the mixing time of a sub-volume of a reactor unit is defined as the time required for achieving a pre-determined degree of homogeneity in said sub-volume of said reactor unit,
finally optionally wherein the mixing time of a sub-volume of a reactor unit is dependent on the volumetric power input.

10. A method of simulating, modelling, monitoring or modifying the conditions of a reactor comprising a liquid composition in a bioreactor or live fermentation process, comprising the steps of:
a. providing the scale-down reactor according to any one of the preceding claims;
b. defining the total reactor volume by reversibly adding a number of inner reactor units (3);
c. defining the number, position and design of the at least one disk (7); and
d. optionally, defining the design of the mixing shaft (5) and one or more impeller (6);
wherein the conditions to be simulated, modelled, monitored or modified are selected from the group consisting of: reaction volume, temperature, pH, gas concentration, metabolite concentration, cellular mass, cellular density, enzymatic kinetics, absorbance, and mixing time, and
wherein the reactor is at least 300 L,
optionally wherein one or more of the conditions is simulated, modelled, monitored or modified in each sub-volume of the scale-down reactor, further optionally wherein one or more operating conditions in the scale-down reactor are equal or similar to said corresponding operating conditions in the reactor, wherein said operating conditions are selected from:
e. Power input value (P/V);
f. aspect ratio;
g. impeller tip speed;
h. mixing time;
i. volumetric mass transfer coefficient; and/or
j. oxygen transfer rate;
wherein P is the Power Input and V is the Volume in the reactor, and wherein P may be calculated according to the formula:
$$P = Po ⋆ ρ ⋆ N ⋆ {D}^{3}$$
wherein V is the volume of the reaction liquid, Po is the Power number for the impeller, p is the fluid density, N is the rotational speed of the impeller and D is the impeller diameter;
wherein the aspect ratio is defined as the inner diameter divided by the inner height of the reactor;
wherein the impeller tip speed may be calculated according to the formula:
Impeller tip speed=π*D*n
wherein D is the impeller diameter and n is the impeller rotation speed;
wherein the mixing time is defined as the time required for achieving a pre-determined degree of homogeneity in the reactor;
wherein the volumetric mass transfer coefficient (k_{L}a) and/or the oxygen transfer rate (OTR) may be calculated according to the formula:
$${k}_{L} a = OTR / ( {C}_{L}^{⋆} - {C}_{L} )$$
wherein C*_{L} is the dissolved oxygen concentration at air saturation and C_{L} is the dissolved oxygen concentration in the liquid phase;
optionally wherein the scale-down reactor is operated in the same mode as the reactor, such as in batch, fed-batch or continuous mode, and further wherein the scale-down reactor is of the same type as the reactor, wherein the type is selected from a stirred tank reactor and a bubble column reactor.

11. Use of a scale-down reactor according to any one of claims 1 to 9 for simulating, modelling, monitoring or modifying the conditions of a reactor, wherein the conditions to be simulated, modelled, monitored or modified are selected from the group consisting of: reaction volume, temperature, pH, gas concentration, metabolite concentration, cellular mass, cellular density, enzymatic kinetics, absorbance, and mixing time,
wherein the reactor is at least 300 L,
optionally wherein the reactor is a bioreactor or a live fermentation process, further optionally wherein one or more operating conditions in the scale-down reactor are equal or similar to said corresponding operating conditions in the reactor, wherein said operating conditions are selected from:
k. Power input value (P/V);
l. aspect ratio;
m. impeller tip speed;
n. mixing time;
o. volumetric mass transfer coefficient; and/or
p. oxygen transfer rate;
wherein P is the Power Input and V is the Volume in the reactor, and wherein P may be calculated according to the formula:
$$P = Po ⋆ ρ ⋆ N ⋆ {D}^{3}$$
wherein V is the volume of the reaction liquid, Po is the Power number for the impeller, ρ is the fluid density, N is the rotational speed of the impeller and D is the impeller diameter;
wherein the aspect ratio is defined as the inner diameter divided by the inner height of the reactor;
wherein the impeller tip speed may be calculated according to the formula:
$$Impeller tip speed = π ⋆ D ⋆ n$$
wherein D is the impeller diameter and n is the impeller rotation speed;
wherein the mixing time is defined as the time required for achieving a pre-determined degree of homogeneity in the reactor;
wherein the volumetric mass transfer coefficient (k_{L}a) and/or the oxygen transfer rate (OTR) may be calculated according to the formula:
$${k}_{L} a = OTR / ( {C}_{L}^{⋆} - {C}_{L} )$$
wherein C*_{L} is the dissolved oxygen concentration at air saturation and C_{L} is the dissolved oxygen concentration in the liquid phase;
optionally wherein the scale-down reactor is operated in the same mode as the reactor, such as in batch, fed-batch or continuous mode, and further wherein the scale-down reactor is of the same type as the reactor, wherein the type is selected from a stirred tank reactor and a bubble column reactor.

12. Use of the stirred tank reactor according to any one of claims 1 to 9 for liquid-liquid extraction, wherein the stirred tank reactor comprises a mixing shaft.

13. A method of adapting a microorganism to the conditions of a reactor, said method comprising:
a. providing the scale-down reactor according to any one of claims 1 to 9;
b. defining the total reactor volume by reversibly adding a number of inner reactor units (3);
c. defining the number, position and design of the at least one disk (7);
d. optionally, defining the design of the mixing shaft (5) and one or more impellers (6); and
e. growing the microorganism in the scale-down reactor for an extended amount of time in order to allow the microorganism to adapt to the conditions of the reactor;
wherein the reactor is at least 300 L, thereby obtaining an adapted microorganism.

14. A system comprising the scale-down reactor according to any one of claims 1 to 9 and a reaction liquid.

15. A method of manufacturing a scale-down reactor, said method comprising:
a. providing
i. a reaction compartment comprising at least two reactor units, wherein said at least two reactor units are reversibly attached to one another via connection means,
wherein the at least two reactor units comprise a top unit (1) and a bottom unit (2), and optionally comprise one or more further inner reactor units (3); wherein said reactor units when attached to one another form a fluid-tight reaction compartment for receiving a reaction liquid therein; wherein one or more of said units comprise one or more inlet fluid apertures (4) and one or more outlet fluid apertures (4),
ii. optionally, a mixing shaft (5) placed vertically in the reaction compartment, said mixing shaft comprising one or more impellers (6), and
iii. at least one disk (7) inserted horizontally between two adjacent reactor units or horizontally within an inner reaction unit, said disk comprising at least one aperture (16) allowing the fluid contained in the adjacent reactor units to pass through it, optionally said disk comprising at least one shaft aperture (8) allowing the mixing shaft to pass through it;
b. assembling said reaction units, disk and optionally said mixing shaft.

16. A method of controlling a scale-down reactor, said method comprising:
a. providing
i. a reaction compartment comprising at least two reactor units, wherein said at least two reactor units are reversibly attached to one another via connection means,
wherein the at least two reactor units comprise a top unit (1) and a bottom unit (2), and optionally comprise one or more further inner reactor units (3); wherein said reactor units when attached to one another form a fluid-tight reaction compartment for receiving a reaction liquid therein; wherein one or more of said units comprise one or more inlet fluid apertures (4) and one or more outlet fluid apertures (4),
ii. optionally, a mixing shaft (5) placed vertically in the reaction compartment, said mixing shaft comprising one or more impellers (6), and
iii. at least one disk (7) inserted horizontally between two adjacent reactor units or horizontally within an inner reaction unit, said disk comprising at least one aperture (16) allowing the fluid contained in the adjacent reactor units to pass through it, optionally said disk comprising at least one shaft aperture (8) allowing the mixing shaft to pass through it;
iv. at least one probe located in at least one of the reactor units, configured to measure at least one property of the reactor, such as pH, dissolved oxygen, temperature, real-time flow cytometry and/or concentration of one or more products present inside the reactor,
b. assembling said reactions units, disk and optionally said mixing shaft,
c. providing a computing unit containing a digital representation of the scale-down reactor,
d. receiving data from the at least one probe, and
e. varying operating conditions of the scale-down reactor until a simulated optimal state provided by the digital representation of the scale-down reactor is achieved.

17. The method according to claim 16, wherein the digital representation of the scale-down reactor is indistinguishable from the scale-down reactor.

18. The method according to any one of claims 16 to 17, wherein the digital representation of the scale-down reactor is a modelling system, such as a digital twin model, a machine learning model or a neural network model.
